(19) United States
(12) Patent Application Publication
(10) Pub. No.: US 2025/0073309 A1
(43) Pub. Date: Mar. 6, 2025

(54) METHODS OF TREATMENT OF PEDIATRIC PUBERTY USING ORAL FORMULATIONS OF LEUPROLIDE

(71) Applicant: Enteris Biopharma, Inc., Boonton, NJ (US)

(72) Inventors: Gary A. Shangold, Califon, NJ (US); Paul P. Shields, North Caldwell, NJ (US); John S. Vrettos, Long Valley, NJ (US)

(73) Assignee: Enteris Biopharma, Inc., Boonton, NJ (US)

(21) Appl. No.: 18/553,017

(22) PCT Filed: Apr. 7, 2022
(86) PCT No.: PCT/US2022/023759

§ 371 Date: Sep. 28, 2023

Related U.S. Application Data
(60) Provisional application No. 63/299,491, filed on Jan. 14, 2022.
(60) Provisional application No. 63/287,801, filed on Dec. 9, 2021.
(60) Provisional application No. 63/172,607, filed on Apr. 8, 2021.

(51) Int. Cl. A61K 38/22; A61K 9/00; A61K 9/20

(52) U.S. Cl. CPC A61K 38/22; A61K 9/0024; A61K 9/0053; A61K 9/2013; A61K 9/2027; A61K 9/2054

(57) ABSTRACT

The present disclosure relates to methods, formulations and dosage regimen for treating pediatric central precocious puberty. The solid oral dosage form of the present disclosure provides an improved alternative to other methods of treatment of central precocious puberty, which are all injectable products. An oral tablet formulation of leuprolide offers a convenient and painless treatment option that allows for flexible dose adjustment or stopping treatment, in contrast to the injectable depot formulations.

### RELATED APPLICATIONS

This application claims the benefit of and priority to U.S. provisional Application Ser. No. 63/172,607, filed Apr. 8, 2021, U.S. provisional Application Ser. No. 63/287,801, filed Dec. 9, 2021, and U.S. provisional Application Ser. No. 63/299,491, filed Jan. 14, 2022, the disclosure of each of which is incorporated herein by reference in their entireties.

### FIELD

The present disclosure relates, in general to methods, formulations and dosage regimen for treating pediatric central precocious puberty.

### BACKGROUND

Oral delivery of active pharmaceutical ingredients is generally the delivery route of choice because it is convenient, relatively easy, and generally painless, resulting in greater patient compliance relative to other modes of delivery. Avoidance of injectable medications is particularly helpful when dealing with the pediatric population, who tend to be even more averse to needles than are adults, and especially for medications that need to be administered over a relatively chronic (long) period of time, measured in months or years, rather than simply days or weeks.

### SUMMARY

Aspects of the disclosure relate to methods of treating a pediatric subject with central precocious puberty (CPP). In some embodiments, the method comprises administering orally to the pediatric subject having CPP a total daily dose of from about 20 mg to about 60 mg leuprolide or a pharmaceutically acceptable salt thereof in a solid dosage form, wherein the pediatric subject has a body weight of less than 25 kg. In some embodiments, the method comprises administering orally to the pediatric subject having CPP a total daily dose of from about 20 mg to about 40 mg leuprolide or a pharmaceutically acceptable salt thereof in a solid dosage form, wherein the pediatric subject has a body weight of less than 25 kg. In some embodiments, the method comprises administering orally to the pediatric subject having CPP a total daily dose of from about 40 mg to about 60 mg leuprolide or a pharmaceutically acceptable salt thereof in a solid dosage form, wherein the pediatric subject has a body weight of less than 25 kg. In some embodiments, the method comprises administering orally to the pediatric subject having CPP a total daily dose of from about 40 mg to about 80 mg leuprolide or a pharmaceutically acceptable salt thereof in a solid dosage form, wherein the pediatric subject has a body weight ranging from 25 kg and 37.5 kg. In some embodiments, the method comprises administering orally to the pediatric subject having CPP a total daily dose of from about 40 mg to about 60 mg leuprolide or a pharmaceutically acceptable salt thereof in a solid dosage form, wherein the pediatric subject has a body weight ranging from 25 kg and 37.5 kg. In some embodiments, the method comprises administering orally to the pediatric subject having CPP a total daily dose of from about 60 mg to about 80 mg leuprolide or a pharmaceutically acceptable salt thereof in a solid dosage form, wherein the pediatric subject has a body weight ranging from 25 kg and 37.5 kg. In some embodiments, the method comprises administering orally to the pediatric subject having CPP a total daily dose from about 60 mg to about 120 mg leuprolide or a pharmaceutically acceptable salt thereof in a solid dosage form, wherein the pediatric subject has a body weight of greater than 37.5 kg. In some embodiments, the method comprises administering orally to the pediatric subject having CPP a total daily dose from about 60 mg to about 90 mg leuprolide or a pharmaceutically acceptable salt thereof in a solid dosage form, wherein the pediatric subject has a body weight of greater than 37.5 kg. In some embodiments, the method comprises administering orally to the pediatric subject having CPP a total daily dose from about 90 mg to about 120 mg leuprolide or a pharmaceutically acceptable salt thereof in a solid dosage form, wherein the pediatric subject has a body weight of greater than 37.5 kg. In some embodiments, the total daily dose is administered once daily. In some embodiments, the total daily dose is administered twice daily. In some embodiments, the once daily dose is administered in the morning before consumption of food. In some embodiments, half of the total daily dose is administered twice daily. In some embodiments, a first and a second half daily doses are administered at about 10 hours to about 14 hours interval. In some embodiments, the first half daily dose of the two doses is administered in the morning before consumption of food. In some embodiments, the second half daily dose of the two doses is administered up to about 6 hours after consumption of food. In some embodiments, the administration reduces mean ratio of bone age to chronological age of the subject at the time of measurement to about 1.4 or lower over a twelve month treatment period. In some embodiments, the administration reduces mean ratio of bone age to chronological age of the subject at the time of measurement to about 1 over about a sixty month treatment period. In some embodiments, the solid dosage form is administered for a time period of about 6 months, of about 12 months, of about 18 months, of about 24 months, or longer. In some embodiments, the solid dosage form is a tablet or a capsule.

In some embodiments, the method of treating a pediatric subject with central precocious puberty comprises administering orally to the pediatric subject having CPP a solid dosage from comprising a therapeutically effective amount of leuprolide or a pharmaceutically acceptable salt thereof, wherein the therapeutically effective amount of the leuprolide or pharmaceutically acceptable salt thereof is from about 20 mg to about 120 mg, and wherein administration reduces mean ratio of bone age to chronological age of the pediatric subject at the time of measurement to about 1.4 or lower over a twelve month treatment period. In some embodiments, the method of treating a pediatric subject with central precocious puberty comprises administering orally to the pediatric subject having CPP a solid dosage form comprising between about 10 mg to about 60 mg of leuprolide or a pharmaceutically acceptable salt thereof, wherein administration of the solid dosage form for a suitable period of time reduces the mean bone age to chronological age of the pediatric subject at the time of measurement to about 1.4 or lower over about a twelve-month treatment period. In some embodiments, the solid dosage form comprises from about 20 mg to about 60 mg leuprolide or a pharmaceutically acceptable salt thereof. In some embodiments, the solid dosage form comprises from about 40 mg to about 60 mg leuprolide or a pharmaceutically acceptable salt thereof. In some embodiments, the solid dosage form comprises from about 50 mg to about 60 mg leuprolide or a pharmaceutically acceptable salt thereof. In some embodiments, the solid dosage form comprises about 60 mg of leuprolide or a pharmaceutically acceptable salt thereof. In some embodiments, the solid dosage form comprises about 50 mg of leuprolide or a pharmaceutically acceptable salt thereof. In some embodiments, the solid dosage form comprises about 45 mg of leuprolide or a pharmaceutically acceptable salt thereof. In some embodiments, the solid dosage form comprises more than about 40 mg and less than about 60 mg of leuprolide acetate. In some embodiments, the solid dosage form is administered once daily. In some embodiments, the solid dosage form is administered twice daily. In some embodiments, the solid dosage form is administered for a time period of about 6 months, of about 12 months, of about 18 months, of about 24 months, or longer. In some embodiments, the administration reduces mean ratio of bone age to chronological age of the pediatric subject at the time of measurement to about 1 over about a sixty month treatment period.

In some embodiments, leuprolide or a pharmaceutically acceptable salt thereof for use in a method of treating a pediatric subject with central precocious puberty (CPP) is provided. In some embodiments, the method comprises administering orally to the pediatric subject having CPP a total daily dose of from about 20 mg to about 60 mg of the leuprolide or pharmaceutically acceptable salt thereof in a solid dosage form, wherein the pediatric subject has a body weight of less than 25 kg. In some embodiments, the method comprises administering orally to the pediatric subject having CPP a total daily dose of from about 20 mg to about 40 mg of the leuprolide or pharmaceutically acceptable salt thereof in a solid dosage form, wherein the pediatric subject has a body weight of less than 25 kg. In some embodiments, the method comprises administering orally to a pediatric subject having CPP a total daily dose of from about 40 mg to about 60 mg of the leuprolide or pharmaceutically acceptable salt thereof in a solid dosage form, wherein the pediatric subject has a body weight of less than 25 kg. In some embodiments, the method comprises administering orally to the pediatric subject having CPP a total daily dose of from about 40 mg to about 80 mg of the leuprolide or pharmaceutically acceptable salt thereof in a solid dosage form, wherein the pediatric subject has a body weight ranging from 25 kg and 37.5 kg. In some embodiments, the method comprises administering orally to the pediatric subject having CPP a total daily dose of from about 40 mg to about 60 mg of the leuprolide or pharmaceutically acceptable salt thereof in a solid dosage form, wherein the pediatric subject has a body weight ranging from 25 kg and 37.5 kg. In some embodiments, the method comprises administering orally to the pediatric subject having CPP a total daily dose of from about 60 mg to about 80 mg of the leuprolide or pharmaceutically acceptable salt thereof in a solid dosage form, wherein the pediatric subject has a body weight ranging from 25 kg and 37.5 kg. In some embodiments, the method comprises administering orally to the pediatric subject having CPP a total daily dose from about 60 mg to about 120 mg of the leuprolide or pharmaceutically acceptable salt thereof in a solid dosage form, wherein the pediatric subject has a body weight of greater than 37.5 kg. In some embodiments, the method comprises administering orally to the pediatric subject having CPP a total daily dose from about 60 mg to about 90 mg of the leuprolide or a pharmaceutically acceptable salt thereof in a solid dosage form, wherein the pediatric subject has a body weight of greater than 37.5 kg. In some embodiments, the method comprises administering orally to the pediatric subject having CPP a total daily dose from about 90 mg to about 120 mg of the leuprolide or pharmaceutically acceptable salt thereof in a solid dosage form, wherein the pediatric subject has a body weight of greater than 37.5 kg. In some embodiments, the method comprises administering orally to the pediatric subject having CPP a solid dosage form comprising a therapeutically effective amount of leuprolide or a pharmaceutically acceptable salt thereof, wherein the therapeutically effective amount of the leuprolide or pharmaceutically acceptable salt thereof is from about 20 mg to about 120 mg, and wherein administration reduces mean ratio of bone age to chronological age of the pediatric subject at the time of measurement to about 1.4 or lower over twelve month treatment period. In some embodiments, the method comprises administering orally to the pediatric subject having CPP a solid dosage form comprising between about 10 mg to about 60 mg of the leuprolide or pharmaceutically acceptable salt thereof, wherein administration of the solid dosage form for a suitable period of time reduces the mean bone age to chronological age of the pediatric subject at the time of measurement to about 1.4 or lower over about a twelve-month treatment period.

In some embodiments, the solid dosage form is a tablet or a capsule. In some embodiments, the solid dosage form is a tablet comprising (a) leuprolide or a pharmaceutically acceptable salt thereof, (b) an absorption enhancer; (c) coated acid particles intermixed with the leuprolide or pharmaceutically acceptable salt thereof, wherein the coating separates the acid from the leuprolide or pharmaceutically acceptable salt thereof in the tablet; (d) an outer layer of an acid-resistant enteric coating effective to transport the tablet through the stomach of the pediatric subject while preventing contact between the leuprolide or pharmaceutically acceptable salt thereof and stomach proteases; and (e) a water soluble barrier layer beneath the outer layer of enteric coating that separates the enteric coating from the coated acid particles. In some embodiments, the coated acid particles comprise coated citric acid particles. In some embodiments, the pharmaceutically acceptable salt of leuprolide is leuprolide acetate. In some embodiments, the absorption enhancer comprises an acyl camitine. In some embodiments, the absorption enhancer comprises lauroyl camitine. In some embodiments, the administration reduces mean ratio of bone age to chronological age of the pediatric subject at the time of measurement to about 1 over about a sixty month treatment period.

### DETAILED DESCRIPTION

It is to be understood that this disclosure is not limited to particular compositions, methods, and experimental conditions described, as such compositions, methods, and conditions may vary. It is also to be understood that the terminology used herein is for purposes of describing particular embodiments only, and is not intended to be limiting, since the scope of the present disclosure will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the disclosure, the preferred methods and materials are now described.

As used herein, the terms “formulation” and “composition” are used interchangeably and refer to a mixture of two or more compounds, elements, or molecules. In some aspects, the terms “formulation” and “composition” may be used to refer to a mixture of one or more active agents with an acid, a permeation enhancer, and optionally additional excipients.

All references to residence times in the gastrointestinal tract (GIT) assume a fasted state. All references to bioavailability (% F) denote absolute bioavailability (relative to intravenous injection), unless otherwise stated.

The terms “API”, “active agent” and “drug” are used interchangeably herein to refer to a therapeutically, and/or prophylactically active compound that has physiologic, pharmaceutical, pharmacological, or therapeutic effect. The term is intended to include the API in any suitable form, such as, e.g., a pharmaceutically acceptable salt, complex, solvate or prodrug thereof, or, if relevant, in any stereoisomer form including any enantiomeric or racemic form, or a combination of any of the above. Examples of APIs include, but are not limited to, a hormone, a peptide, a small molecule, or prodrug thereof.

As used herein, the terms “dosage form”, “pharmaceutical composition” and “pharmaceutical formulations” are used interchangeably herein to refer to physically discrete units suitable as unitary dosages for human subjects and other mammals, each unit containing a predetermined quantity of therapeutic agent calculated to produce the desired onset, tolerability, and therapeutic effects, in association with one or more suitable pharmaceutical excipients. Methods for preparing such dosage forms are known or will be apparent to those skilled in the art. The dosage forms of the present disclosure may take the form of a solid dosage form such as a tablet, pill, capsule, or the like. In some embodiments, the solid dosage form is a tablet. In other embodiments, the solid dosage form is a capsule. In some embodiments, the dosage form is a solid delayed release dosage form, for example, but not limited to, a delayed or extended release tablet or a delayed or extended release capsule. In some embodiments, the tablet or capsule is enteric coated. In some embodiments, the solid dosage form is tablet that is enteric coated. In other embodiments, the dosage form is comprised of minitablets, granules, or multiparticles filled in a capsule or sachet. In some embodiments, the minitablets, granules or multiparticles are enteric coated, or coated for taste-masking.

As used herein, an “effective amount” or a “therapeutically effective amount” of an API refers to a non-toxic, but sufficient amount of the API, to achieve therapeutic results in treating a condition for which the API is known to be effective. It is understood that various biological factors may affect the ability of a substance to perform its intended task.

As used herein, “about” and “approximately” generally mean an acceptable degree of error for the quantity measured given the nature or precision of the measurements. Exemplary degrees of error are within 20 percent (%), typically, within 10%, and more typically, within 5% of a given range of values. The term “substantially” means more than 50%, more than 80%, or more than 90% or 95%.

The terms “polypeptide,” “peptide” and “protein” are used interchangeably herein to refer to a polymer of amino acid residues having at least one peptide bond. The terms apply to naturally occurring amino acid polymers as well as amino acid polymers in which one or more amino acid residues are a non-natural amino acid. Additionally, such “polypeptides,” “peptides” and “proteins” include amino acid chains of any length, including full length proteins, wherein the amino acid residues are linked by covalent peptide bonds. In some embodiments, the peptides can be cyclic peptides. In some embodiments, the API is a peptidomimetic. As used herein the term “peptidomimetic” refers to a small protein-like chain designed to mimic a peptide.

A “non-natural amino acid” refers to an amino acid that is not one of the 20 common amino acids, namely alanine, cysteine, aspartic acid, glutamic acid, phenylalanine, lysine, histidine, isoleucine, lysine, leucine, methionine, asparagine, proline, glutamine, arginine, serine, threonine, valine, tryptophan, and tyrosine, or pyrolysine or selenocysteine. Other terms that may be used synonymously with the term “non-natural amino acid” is “non-naturally encoded amino acid,” “unnatural amino acid,” “non-naturally-occurring amino acid.” The term “non-natural amino acid” includes, but is not limited to, amino acids which occur naturally by modification of a naturally encoded amino acid (including but not limited to, the 20 common amino acids or pyrrolysine and selenocysteine) but are not themselves incorporated into a growing polypeptide chain by the translation complex. Examples of naturally-occurring amino acids that are not naturally-encoded include, but are not limited to, N-acetylglucosaminyl-L-serine, N-acetylglucosaminyl-L-threonine, and O-phosphotyrosine. Additionally, the term “non-natural amino acid” includes, but is not limited to, amino acids which do not occur naturally and may be obtained synthetically or may be obtained by modification of non-natural amino acids. Non-natural amino acids can include amino acids containing the D-isomer configuration since most proteins are comprised primarily or entirely of amino acids in the L-isomer configuration. Peptides containing non-natural amino acids, such as D-amino acids and those including substituted side chains can exhibit improved stability in the gastrointestinal tract as a result of reduced proteolysis. In addition to natural amino acids, the amino acids may be D-amino acids or unnatural amino acids, some examples of which are discussed infra. The molecular structure may further include other substituents or modifications. Some peptides may be amidated at locations that are not amidated in nature, or may be otherwise modified.

Various GnRH agonists and antagonists are currently either approved or in clinical investigations designed to support marketing applications for multiple clinical indications. These include both benign and malignant conditions which are stimulated and/or sustained by circulating levels of gonadal steroid hormones: estrogens (and possibly) progestogens in females, and androgens (predominantly testosterone) in males. The malignant conditions which are currently treated are predominantly prostatic carcinoma (males), and less commonly hormone-dependent breast carcinoma (mostly females, but occasionally also in males). In these conditions, the GnRH analogue helps prevent recurrences or further spread of the cancers by eliminating, via suppression, production of the gonadal sex steroid hormones; the treatments can be a follow-up, adjunct, or alternative to initial surgical treatment and/or chemotherapy. Treatment, once initiated, is continuous for a period of years, typically using a depot formulation that is injected either monthly, or every 3 or 6 months.

Benign conditions treated with these compounds include: endometriosis, uterine leiomyomata (benign fibroid tumors) in females; central precocious puberty (both sexes, though approximately 20 times more common in girls than in boys). The benefits of treating endometriosis include treatment of related pelvic pain caused by ectopic endometrial implants and concomitant cytokine release, as well as potential mitigation of endometriosis-mediated infertility. Endometriosis is typically treated for ˜6 months (12 months maximally), using depot injections given every month, 3 months, or 6 months. The benefits of treating uterine fibroids are shrinkage of the fibroids with resultant mitigation of pressure on anatomically adjacent organs like the bladder and rectum, reduction in heavy or prolonged uterine bleeding (either during or between normal menstrual periods), and improvement in anemia which results from the heavy bleeding; reduction of pain from fibroids can also result from treatment. Fibroids are typically treated for 2-4 months, most often prior to surgical removal of the fibroids, in order to stabilize the patient's hemoglobin prior to surgery, and may occasionally be even longer as an alternative to surgical therapy.

Instances in which both agonists and antagonists are used clinically is as part of a regimen of “controlled ovarian stimulation” given as part of a treatment plan for In Vitro Fertilization (IVF) and Embryo Transfer for many kinds of infertility. In this case, the GnRH analogue is used both to enhance the ability to maximally stimulate the ovaries to produce multiple mature follicles which can be the source for obtaining fertilizable oocytes, as well as in preventing the premature occurrence of an endogenous LH surge, which, if it occurs, can lead to ovulation and loss of the eggs into the pelvic cavity before the clinician can harvest the oocytes directly from the unruptured ovarian follicles. In the IVF setting, GnRH antagonists are used in 1-2 different stimulation protocols, while GnRH agonists are used either in a) the ‘long’ protocol, beginning 2 weeks prior to the cycle of stimulation, b) the ‘ultrashort flare’ protocol, beginning concomitantly with stimulation, or c) as an ovulation trigger, given 36 hours prior to intended oocyte harvest. Depending upon the specific protocol, the treatment with GnRH analogs can last for 5 days to 3 weeks, or only via a single injection if as an ovulation trigger. It is usually given as a daily subcutaneous injection in the IVF stimulation protocol setting.

### Precocious Puberty

There is a wide range of ages at which individuals normally start puberty. Precocious Puberty (PP) is characterized by early onset of pubertal changes to a child of at least 2 years of age. PP is further divided into two classifications: Peripheral Precocious Puberty (PPP) or Central Precocious Puberty (CPP).

Precocious puberty may be a complication of McCune-Albright syndrome or congenital adrenal hyperplasia (CAH)—conditions that involve abnormal production of the male hormones (androgens). In rare cases, precocious puberty may also be associated with hypothyroidism.

PPP is defined by early sexual development prompted by sex steroids independent of pituitary gonadotropins from abnormal endogenous or exogenous sources such as disease or environmental exposure. CPP is defined by early sexual development prompted by production and release of pituitary gonadotropins and/or gonadal sex steroids, as a result of hormonal stimulation from normal endogenous sources including the hypothalamus and pituitary.

It is estimated that 20 out of every 10,000 girls and 1 out of every 10,000 boys suffer from central precocious puberty or premature puberty. This condition is evident when girls under the age of eight years and boys under the age of nine years develop signs of sexual maturity, such as the early onset of secondary sexual characteristics, increase in growth rate, and advancement of skeletal age beyond chronological age. Signs or symptoms of CPP include, but are not limited to, the development of secondary sex characteristics such as breasts, testicle growth, or pubic hair.

Initial screening tests usually include bone age, measurement of luteinizing hormone (LH), follicle-stimulating hormone (FSH), testosterone, dehydroepiandrosterone sulfate (DHEA-S), estradiol, progesterone, 17-OH progesterone levels, and thyroid function tests. Magnetic resonance imaging is to be performed in all cases of CPP, especially in males, to rule out a hypothalamic lesion.

Untreated precocious puberty usually leads to short stature via premature closure of the skeletal epiphyseal growth plates, and can also cause significant emotional and behavioral issues.

CPP can be treated using injections of leuprolide-microsphere injectable formulations intramuscularly in a 1.0 or 1.5 mL volume, with varying and adjustable dosage (7.5, 11.25, or 15 mg for 1 month; 11.25 or 30 mg for 3 month, LUPRON DEPOT-PED) based upon the child's weight and/or the child's clinical response to the formulation.

Approved leuprolide treatments for CPP in children have limitations and disadvantages, including but not limited to patient discomfort, patient non-compliance, and patient-to-patient variability. It is usually treated either with a monthly, 3-monthly, or 6-monthly injectable depot of leuprolide, or with a subcutaneous surgical implant delivering histrelin, another GnRH agonist, that must be renewed every 12-18 months. For injectable depot, polymer-based microspheres leuprolide formulations must be prepared and administered by a physician to ensure correct dosing. Up to 2 mL of the polymer-based microspheres leuprolide formulations is administered to children via deep, intramuscular injection. Such injections are painful and difficult to perform. The injections are occasionally associated with inflammatory reactions at the injection site, and infrequently with abscess formation at the site of the depot injection. Polymer-based microspheres leuprolide formulations provide continuous release of leuprolide agonist for, for example, 1, 3, or 6 months and therefore removal/disposal of the formulation cannot be performed if rapid discontinuation of treatment is desired.

The benefits of treating precocious puberty include: arrest of premature growth spurt and the often resultant premature closure of the epiphyseal growth plates which usually leads to a significant reduction in ultimate adult height; also, mitigation of psycho-social pressures related to the early and inappropriate onset of pubertal symptoms in children whose chronological age is often only between 3-8 years. Treatment of precocious puberty is usually begun once the diagnosis is made and continued until the child is of an age which is appropriate for puberty.

Oral formulations and methods of treatment of the present disclosure have the advantage to be an effective, more tolerable treatment by avoiding the difficulty and pain associated with the administration of medication by injection. The benefits of oral administration include, but are not limited to, that (1) oral dosing avoids the need for painful injection of the depot, and (2) therapy can be stopped by discontinuing the oral medication, which is not an option with the depot. Also, occasional injection site reactions (inflammation, even abscess formation) can be avoided by eliminating the need for injectable route of administration. Such oral formulations and methods of treatment are therefore highly advantageous over current CPP treatment options.

Without being bound to the theory, children require higher doses of GnRH agonists to treat CPP than the adult doses needed for indications such as endometriosis and uterine fibroids. Approved doses of leuprolide acetate as depot injections are 3.75 mg/month and 11.25 mg q3 months for endometriosis and fibroids; 7.5 mg/month, 22.5 mg q3 months, 30 mg q4 months, and 45 mg q6 months for prostate cancer; 7.5, 11.25, and 15 mg/month, 11.25 and 30 mg q3 months for Central Precocious Puberty. The three different doses are intended for children of variable body weights. There is also a 1 mg daily subcutaneous formulation of leuprolide (used mostly now for IVF), and a 6-month 45 mg subcutaneous injectable formulation of leuprolide acetate approved for central precocious puberty.

Administration of GnRH agonists produces suppression following an initial period (1-2 weeks) of stimulation or flare, by a combination of GnRH membrane receptor downregulation and several post-receptor cellular desensitization mechanisms.

In some embodiments, delivery of GnRH or GnRH agonists results in chronic stimulation of GnRH receptors in the pituitary, which after an initial transient increase or stimulation, leads to a subsequent downregulation of GnRH receptor density. Downregulation of GnRH receptors reduces GnRH-dependent secretion of gonadotropins, including but not limited to luteinizing hormone (LH) and follicle-stimulating hormone (FSH), which are key drivers of normal development during puberty. Reduction in gonadotropin secretion results in the slowing and/or partial or significant reversal of the early onset of pubertal symptoms associated with CPP.

GnRH antagonists produce immediate suppression, mediated by direct competitive blockade of the pituitary gonadotrope membrane receptor for GnRH.

Whether using an agonist or antagonist, the goal is inhibition of production of pituitary gonadotropins (Luteinizing Hormone and Follicle Stimulating Hormone), the absence of which then causes failure to produce ovarian estrogens (and progesterone) or testicular testosterone.

Aspects of the disclosure relate to methods of treatment of CPP and comprise administering orally a solid dosage form. In some embodiments, the solid dosage form is a tablet. In some embodiments, the solid dosage is a tablet comprising a gonadotrophin releasing hormone (GnRH) agonist or analogue thereof. In some embodiments, the solid dosage is a tablet comprising leuprolide. In some embodiments, the solid dosage is a tablet comprising leuprolide acetate. In some embodiments, the solid dosage form can be administered one, twice, or more times daily.

In some embodiments, the gonadotrophin releasing hormone (GnRH) agonist or analogue thereof include, but is not limited to, leuprolide, goserelin, histrelin, buserelin, nafarelin, triptorelin or pharmaceutically acceptable salt thereof. In some embodiments, the hormone or analogue thereof is selected from a gonadotrophin releasing hormone (GnRH) agonist or antagonist including, but not limited to, triptorelin, leuprorelin, goserelin, nafarelin, ganirelix, and cetrorelix or pharmaceutically acceptable salt thereof. As used herein, the term “leuprolide” refers to leuprorelin, derivatives thereof, or pharmaceutically acceptable salt thereof. Derivatives of leuprolide) include, but are not limited to leuprolide 6NMeDLeu, leuprolide 8NMeArg, leuprolide 3NMeTNal, leuprolide 2 Phe, leuprolide 2NMeHis, leuprolide 2NMePhe, leuprolide TOSarNH2, leuprolide-ethyl-D5, leuprolide 5NMeTyr, leuprolide 7NMeLeu, leuprolide 4NMeSer, and leuprolide 3-1Nal. Pharmaceutically acceptable salts of leuprolide include, but are not limited to, leuprolide acetate, leuprolide monoacetate, leuprolide oleate, leuprolide palmitate leuprolide mesylate, leuprolide trifluoracetic acid (TFA), leuprolide trifluoroacetate, leuprolide (5-9), (D-His2)-leuprolide trifluoracetic acid (TFA), leuprolide hydrochloric acid (HCL), leuprolide-D5 acetate, and leuprolide (L-Leu).

In some embodiments, the solid dosage form is leuprolide acetate. Leuprolide acetate is a synthetic nonapeptide agonist analog of luteinizing hormone-releasing factor. Leuprolide acetate is commercially available (for example, from BACHEM, Torrence, Calif.).

### Methods of Treatment

Some aspects of the disclosure relating to methods of treating a subject are disclosed. In some embodiments, the subject is a pediatric patient who has CPP. As used herein, the term “pediatric patient” is defined as any child ages 2 to 12 years old. In some embodiments the pediatric patient is 2 years old or older. Pediatric patient with CPP is defined as male child about 2 to about 9 years old or female child about 2 to about 8 years old, which display pubertal signs or symptoms associated with CPP, such as abnormal gonadotropin and/or sex hormone levels, and/or secondary sexual characteristics.

In some embodiments, the method of treating comprises administering to the pediatric subject in need thereof a solid oral dosage form comprising a therapeutically effective amount of a gonadotrophin releasing hormone (GnRH) agonist or analogue thereof. In some embodiments, the method of treating comprises administering to the pediatric subject in need thereof a solid oral dosage form comprising a therapeutically effective amount of leuprolide or the pharmaceutically acceptable salt thereof. In some embodiments, gonadotrophin releasing hormone (GnRH) agonist or analogue thereof for use in a method of treating a pediatric subject with central precocious puberty (CPP) is disclosed herein. In some embodiments, leuprolide or a pharmaceutically acceptable salt thereof for use in a method of treating a pediatric subject with central precocious puberty (CPP) is disclosed herein.

In some embodiments, the amount of leuprolide or the pharmaceutically acceptable salt thereof (e.g. leuprolide acetate) in the solid dosage form is from about 10 mg to about 60 mg. In some embodiments, the amount of leuprolide or the pharmaceutically acceptable salt thereof (e.g. leuprolide acetate) in the solid dosage form can be 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 mg.

In some embodiments, the amount of leuprolide or the pharmaceutically acceptable salt thereof (e.g. leuprolide acetate) in the solid dosage form is from about 40 mg to about 60 mg. In some embodiments, the amount of leuprolide or the pharmaceutically acceptable salt thereof (e.g. leuprolide acetate) in the solid dosage form is from about 45 mg to about 60 mg. In some embodiments, the amount of leuprolide or the pharmaceutically acceptable salt thereof (e.g. leuprolide acetate) in the solid dosage form is from about 50 mg to about 60 mg. In some embodiments, the amount of leuprolide or the pharmaceutically acceptable salt thereof (e.g. leuprolide acetate) in the solid dosage form is from about 55 mg to about 60 mg. In some embodiments, the amount of leuprolide or the pharmaceutically acceptable salt thereof (e.g. leuprolide acetate) in the solid dosage form is from about 45 mg to about 55 mg. In some embodiments, the amount of leuprolide or the pharmaceutically acceptable salt thereof (e.g. leuprolide acetate) in the solid dosage form is from about 45 mg to about 50 mg. In some embodiments, the amount of leuprolide or the pharmaceutically acceptable salt thereof (e.g. leuprolide acetate) in the solid dosage form is from about 50 mg to about 55 mg. In some embodiments, the amount of leuprolide or the pharmaceutically acceptable salt thereof in the solid dosage form can be 40, 41, 42, 43, 44, 45, 46, 47, 48, 49, 50, 51, 52, 53, 54, 55, 56, 57, 58, 59 or 60 mg. For example, the solid dosage form comprises 40, 41, 42, 43, 44, 45, 46, 47, 48, 49, 50, 51, 52, 53, 54, 55, 56, 57, 58, 59 or 60 mg leuprolide acetate.

In some embodiments, the solid oral dosage form is administered once or twice daily. In some embodiments, the solid oral dosage form is administered twice daily approximately 10, 11, 12, 13 or 14 hours apart.

In some embodiments, the total dose of about 10 mg to about 120 mg of leuprolide, such as leuprolide acetate, is administered daily. In some embodiments, the total dose of about 20 mg to about 120 mg of leuprolide, such as leuprolide acetate, is administered daily. In some embodiments, the total dose of about 30 mg to about 120 mg of leuprolide, such as leuprolide acetate, is administered daily. In some embodiments, the amount of leuprolide or the pharmaceutically acceptable salt thereof administered daily is from about 40 mg to about 120 mg. In some embodiments, the amount of leuprolide or the pharmaceutically acceptable salt thereof administered daily is from about 80 mg to about 120 mg. In some embodiments, the amount of leuprolide or the pharmaceutically acceptable salt thereof administered daily is from about 90 mg to about 120 mg. In some embodiments, the amount of leuprolide or the pharmaceutically acceptable salt thereof administered daily is from about 100 mg to about 120 mg. In some embodiments, the amount of leuprolide or the pharmaceutically acceptable salt thereof administered daily is from about 110 mg to about 120 mg. In some embodiments, the amount of leuprolide or the pharmaceutically acceptable salt thereof administered daily is from about 40 mg to about 50 mg, from about 40 mg to about 60 mg, from about 40 mg to about 70 mg, from about 40 mg to about 80 mg, from about 40 mg to about 90 mg, from about 40 mg to about 100 mg, from about 40 mg to about 110 mg, from about 40 mg to about 120 mg. In some embodiments, the amount of leuprolide or the pharmaceutically acceptable salt thereof administered daily is from about 45 mg to about 50 mg, from about 45 mg to about 60 mg, from about 45 mg to about 70 mg, from about 45 mg to about 80 mg, from about 45 mg to about 90 mg, from about 45 mg to about 100 mg, from about 45 mg to about 110 mg, or from about 45 mg to about 120 mg. In some embodiments, the amount of leuprolide or the pharmaceutically acceptable salt thereof administered daily is from about 50 mg to about 60 mg, from about 50 mg to about 70 mg, from about 50 mg to about 80 mg, from about 50 mg to about 90 mg, from about 50 mg to about 100 mg, from about 50 mg to about 110 mg, or from about 50 mg to about 120 mg. In some embodiments, the amount of leuprolide or the pharmaceutically acceptable salt thereof administered daily is from about 60 mg to about 70 mg, from about 60 mg to about 80 mg, from about 60 mg to about 90 mg, from about 60 mg to about 100 mg, from about 60 mg to about 110 mg, or from about 60 mg to about 120 mg. In some embodiments, the amount of leuprolide or the pharmaceutically acceptable salt thereof administered daily is from about 70 mg to about 80 mg, from about 70 mg to about 90 mg, from about 70 mg to about 100 mg, from about 70 mg to about 110 mg, or from about 70 mg to about 120 mg. In some embodiments, the amount of leuprolide or the pharmaceutically acceptable salt thereof administered daily is from about 80 mg to about 90 mg, from about 80 mg to about 100 mg, from about 80 mg to about 110 mg, or from about 80 mg to about 120 mg. In some embodiments, the amount of leuprolide or the pharmaceutically acceptable salt thereof administered daily is from about 90 mg to about 100 mg, from about 90 mg to about 110 mg, or from about 90 mg to about 120 mg. In some embodiments, the amount of leuprolide or the pharmaceutically acceptable salt thereof administered daily is from about 100 mg to about 110 mg, or from about 100 mg to about 120 mg. In some embodiments, the amount of leuprolide or the pharmaceutically acceptable salt thereof in the solid dosage form is from about 110 mg to about 120 mg.

In some embodiments, the total dose of about 40, 41, 42, 43, 44, 45, 46, 47, 48, 49, 50, 51, 52, 53, 54, 55, 56, 57, 58, 59, 60, 65, 70, 75, 80, 85, 90, 95, 100, 105, 110, 115, 120 mg of leuprolide, such as leuprolide acetate, is administered daily.

In some embodiments, the solid dosage forms can be administered as a single daily dose (QD) or in divided doses (BID). In some embodiments, half of the total daily dose is administered twice daily. In some embodiments, about 20 mg to about 120 mg of leuprolide can be administered daily, given potentially as either single (QD) daily doses (about 20 mg QD-about 120 mg QD) or possibly in divided (BID) doses (about 10 mg BID-about 60 mg BID).

In some embodiments, about 40-120 mg can be administered as a single daily dose (e.g. about 40, 60, 80, and 120 mg or any amount therebetween QD). In other embodiments, about 20-60 mg can be administered twice daily (e.g. about 20, 30, 40, and 60 mg or any amount therebetween BID).

In some embodiments, a total dose of from about 20 mg to about 40 mg is administered daily to pediatric subjects in need thereof having a body weight less than 25 kg. In some embodiments, a total dose of from about 25 mg to about 40 mg is administered daily to pediatric subjects in need thereof having a body weight less than 25 kg. In some embodiments, a total dose of from about 30 mg to about 40 mg is administered daily to pediatric subjects in need thereof having a body weight less than 25 kg. In some embodiments, a total dose of from about 35 mg to about 40 mg is administered daily to pediatric subjects in need thereof having a body weight less than 25 kg. In some embodiments, a total dose of from about 20 mg to about 25 mg is administered daily to pediatric subjects in need thereof having a body weight less than 25 kg. In some embodiments, a total dose of from about 20 mg to about 30 mg is administered daily to pediatric subjects in need thereof having a body weight less than 25 kg. In some embodiments, a total dose of from about 20 mg to about 35 mg is administered daily to pediatric subjects in need thereof having a body weight less than 25 kg. In some embodiments, a total dose of from about 30 mg to about 35 mg is administered daily to pediatric subjects in need thereof having a body weight less than 25 kg. In some embodiments, the total dose can be administered as a single daily dose or as a divided dose (e.g. from about 10 mg to about 20 mg (or any amount therebetween) twice daily).

In some embodiments, a total dose of from about 40 mg to about 60 mg is administered daily to pediatric subjects in need thereof having a body weight less than 25 kg. In some embodiments, from about 45 mg to about 60 mg is administered daily to pediatric subjects in need thereof having a body weight less than 25 kg. In some embodiments, from about 50 mg to about 60 mg is administered daily to pediatric subjects in need thereof having a body weight less than 25 kg. In some embodiments, from about 55 mg to about 60 mg is administered daily to pediatric subjects in need thereof having a body weight less than 25 kg. In some embodiments, from about 45 mg to about 50 mg is administered daily to pediatric subjects in need thereof having a body weight less than 25 kg. In some embodiments, from about 45 mg to about 55 mg is administered daily to pediatric subjects in need thereof having a body weight less than 25 kg. In some embodiments, from about 40 mg to about 45 mg is administered daily to pediatric subjects in need thereof having a body weight less than 25 kg. In some embodiments, the total dose can be administered as a single daily dose or as a divided dose (e.g. from about 20 mg to about 30 mg (or any amount therebetween) twice daily). For example, a solid dosage form comprising 25 mg of leuprolide or the pharmaceutically acceptable salt thereof can be administered twice daily to a pediatric subjects in need thereof having a body weight less than 25 kg or a solid dosage form comprising 50 mg of leuprolide or the pharmaceutically acceptable salt thereof can be administered once daily to a pediatric subjects in need thereof having a body weight less than 25 kg.

In some embodiments, a total dose of from about 40 mg to about 80 mg is administered daily to pediatric subjects in need thereof having a body weight between 25 kg and 37.5 kg. In some embodiments, from about 45 mg to about 80 mg is administered daily to pediatric subjects in need thereof having a body weight less than 25 kg. In some embodiments, from about 50 mg to about 80 mg is administered daily to pediatric subjects in need thereof having a body weight between 25 kg and 37.5 kg. In some embodiments, from about 55 mg to about 80 mg is administered daily to pediatric subjects in need thereof having a body weight between 25 kg and 37.5 kg. In some embodiments, from about 60 mg to about 80 mg is administered daily to pediatric subjects in need thereof having a body weight between 25 kg and 37.5 kg. In some embodiments, from about 65 mg to about 80 mg is administered daily to pediatric subjects in need thereof having a body weight between 25 kg and 37.5 kg. In some embodiments, from about 70 mg to about 80 mg is administered daily to pediatric subjects in need thereof having a body weight between 25 kg and 37.5 kg. In some embodiments, from about 75 mg to about 80 mg is administered daily to pediatric subjects in need thereof having a body weight between 25 kg and 37.5 kg. In some embodiments, from about 40 mg to about 75 mg is administered daily to pediatric subjects in need thereof having a body weight less than 25 kg. In some embodiments, from about 45 mg to about 75 mg is administered daily to pediatric subjects in need thereof having a body weightless than 25 kg. In some embodiments, from about 50 mg to about 75 mg is administered daily to pediatric subjects in need thereof having a body weight between 25 kg and 37.5 kg. In some embodiments, from about 55 mg to about 75 mg is administered daily to pediatric subjects in need thereof having a body weight between 25 kg and 37.5 kg. In some embodiments, from about 60 mg to about 75 mg is administered daily to pediatric subjects in need thereof having a body weight between 25 kg and 37.5 kg. In some embodiments, from about 65 mg to about 75 mg is administered daily to pediatric subjects in need thereof having a body weight between 25 kg and 37.5 kg. In some embodiments, from about 70 mg to about 75 mg is administered daily to pediatric subjects in need thereof having a body weight between 25 kg and 37.5 kg. In some embodiments, from about 40 mg to about 70 mg is administered daily to pediatric subjects in need thereof having a body weight less than 25 kg. In some embodiments, from about 45 mg to about 70 mg is administered daily to pediatric subjects in need thereof having a body weight less than 25 kg. In some embodiments, from about 50 mg to about 70 mg is administered daily to pediatric subjects in need thereof having a body weight between 25 kg and 37.5 kg. In some embodiments, from about 55 mg to about 70 mg is administered daily to pediatric subjects in need thereof having a body weight between 25 kg and 37.5 kg. In some embodiments, from about 60 mg to about 70 mg is administered daily to pediatric subjects in need thereof having a body weight between 25 kg and 37.5 kg. In some embodiments, from about 65 mg to about 70 mg is administered daily to pediatric subjects in need thereof having a body weight between 25 kg and 37.5 kg. In some embodiments, from about 40 mg to about 65 mg is administered daily to pediatric subjects in need thereof having a body weight less than 25 kg. In some embodiments, from about 45 mg to about 65 mg is administered daily to pediatric subjects in need thereof having a body weight less than 25 kg. In some embodiments, from about 50 mg to about 65 mg is administered daily to pediatric subjects in need thereof having a body weight between 25 kg and 37.5 kg. In some embodiments, from about 55 mg to about 65 mg is administered daily to pediatric subjects in need thereof having a body weight between 25 kg and 37.5 kg. In some embodiments, from about 60 mg to about 65 mg is administered daily to pediatric subjects in need thereof having a body weight between 25 kg and 37.5 kg. In some embodiments, the total dose can be administered as a single daily dose or as a divided dose (e.g. from about 20 mg to about 40 mg (or any amount therebetween) twice daily).

In some embodiments, a total dose of from about 40 mg to about 60 mg is administered daily to pediatric subjects in need thereof having a body weight between 25 kg and 37.5 kg. In some embodiments, the total dose can be administered as a single daily dose or as a divided dose (e.g. from about 20 mg to about 30 mg (or any amount therebetween) twice daily). In some embodiments, from about 45 mg to about 60 mg is administered daily to pediatric subjects in need thereof having a body weight less than 25 kg. In some embodiments, from about 50 mg to about 60 mg is administered daily to pediatric subjects in need thereof having a body weight between 25 kg and 37.5 kg. In some embodiments, from about 55 mg to about 60 mg is administered daily to pediatric subjects in need thereof having a body weight between 25 kg and 37.5 kg. In some embodiments, from about 40 mg to about 55 mg is administered daily to pediatric subjects in need thereof having a body weight between 25 kg and 37.5 kg. In some embodiments, from about 45 mg to about 55 mg is administered daily to pediatric subjects in need thereof having a body weight between 25 kg and 37.5 kg. In some embodiments, from about 50 mg to about 55 mg is administered daily to pediatric subjects in need thereof having a body weight between 25 kg and 37.5 kg. In some embodiments, from about 40 mg to about 50 mg is administered daily to pediatric subjects in need thereof having a body weight between 25 kg and 37.5 kg. In some embodiments, from about 45 mg to about 50 mg is administered daily to pediatric subjects in need thereof having a body weight between 25 kg and 37.5 kg. In some embodiments, from about 40 mg to about 45 mg is administered daily to pediatric subjects in need thereof having a body weight between 25 kg and 37.5 kg.

In some embodiments, from about 60 mg to about 80 mg is administered daily to pediatric subjects in need thereof having a body weight between 25 kg and 37.5 kg. In some embodiments, from about 65 mg to about 80 mg is administered daily to pediatric subjects in need thereof having a body weight between 25 kg and 37.5 kg. In some embodiments, from about 70 mg to about 80 mg is administered daily to pediatric subjects in need thereof having a body weight between 25 kg and 37.5 kg. In some embodiments, from about 60 mg to about 75 mg is administered daily to pediatric subjects in need thereof having a body weight between 25 kg and 37.5 kg. In some embodiments, from about 65 mg to about 75 mg is administered daily to pediatric subjects in need thereof having a body weight between 25 kg and 37.5 kg. In some embodiments, from about 70 mg to about 75 mg is administered daily to pediatric subjects in need thereof having a body weight between 25 kg and 37.5 kg. In some embodiments, from about 60 mg to about 70 mg is administered daily to pediatric subjects in need thereof having a body weight between 25 kg and 37.5 kg. In some embodiments, from about 65 mg to about 70 mg is administered daily to pediatric subjects in need thereof having a body weight between 25 kg and 37.5 kg. In some embodiments, from about 60 mg to about 65 mg is administered daily to pediatric subjects in need thereof having a body weight between 25 kg and 37.5 kg. In some embodiments, from about 75 mg to about 80 mg is administered daily to pediatric subjects in need thereof having a body weight between 25 kg and 37.5 kg. In some embodiments, the total dose can be administered as a single daily dose or as a divided dose (e.g. from about 30 mg to about 40 mg (or any amount therebetween) twice daily). For example, a solid dosage form comprising 35 mg of leuprolide or the pharmaceutically acceptable salt thereof can be administered twice daily to a pediatric subjects in need thereof having a body weight having a body weight between 25 kg and 37.5 kg or a solid dosage form comprising 70 mg of leuprolide or the pharmaceutically acceptable salt thereof can be administered once daily to a pediatric subjects in need thereof having a body weight between 25 kg and 37.5 kg.

In some embodiments, from about 60 mg to about 120 mg is administered daily to pediatric subjects in need thereof having a body weight greater than 37.5 kg. In some embodiments, from about 60 mg to about 90 mg is administered daily to pediatric subjects in need thereof having a body weight greater than 37.5 kg. In some embodiments, from about 60 mg to about 80 mg is administered daily to pediatric subjects in need thereof having a body weight greater than 37.5 kg. In some embodiments, from about 60 mg to about 70 mg is administered daily to pediatric subjects in need thereof having a body weight greater than 37.5 kg. In some embodiments, from about 70 mg to about 90 mg is administered daily to pediatric subjects in need thereof having a body weight greater than 37.5 kg. In some embodiments, from about 70 mg to about 80 mg is administered daily to pediatric subjects in need thereof having a body weight greater than 37.5 kg. In some embodiments, from about 90 mg to about 120 mg is administered daily to pediatric subjects in need thereof having a body weight greater than 37.5 kg. In some embodiments, from about 100 mg to about 120 mg is administered daily to pediatric subjects in need thereof having a body weight greater than 37.5 kg. In some embodiments, from about 110 mg to about 120 mg is administered daily to pediatric subjects in need thereof having a body weight greater than 37.5 kg. For example, a solid dosage form comprising 55 mg of leuprolide or the pharmaceutically acceptable salt thereof can be administered twice daily to a pediatric subjects in need thereof having a body weight greater than 37.5 kg or a solid dosage form comprising 110 mg of leuprolide or the pharmaceutically acceptable salt thereof can be administered once daily to a pediatric subjects in need thereof having a body weight greater than 37.5 kg. In some embodiments, the total dose can be administered as a single daily dose or as a divided dose (e.g. from about 30 mg to about 45 mg (or any amount therebetween) twice daily or from about 45 mg to about 60 mg (or any amount therebetween) twice daily).

In some embodiments, from about 80 mg to about 120 mg is administered daily to pediatric subjects in need thereof having a body weight greater than 37.5 kg. In some embodiments, from about 90 mg to about 120 mg is administered daily to pediatric subjects in need thereof having a body weight greater than 37.5 kg. In some embodiments, from about 100 mg to about 120 mg is administered daily to pediatric subjects in need thereof having a body weight greater than 37.5 kg. In some embodiments, from about 110 mg to about 120 mg is administered daily to pediatric subjects in need thereof having a body weight greater than 37.5 kg. In some embodiments, the total dose can be administered as a single daily dose or as a divided dose (e.g. from about 40 mg to about 60 mg (or any amount therebetween) twice daily). For example, a solid dosage form comprising 55 mg of leuprolide or the pharmaceutically acceptable salt thereof can be administered twice daily to a pediatric subjects in need thereof having a body weight greater than 37.5 kg or a solid dosage form comprising 110 mg of leuprolide or the pharmaceutically acceptable salt thereof can be administered once daily to a pediatric subjects in need thereof having a body weight greater than 37.5 kg.

In some embodiments, the solid dosage form is administered without food. In some embodiments, the administration without food can occur when the patient is in a fasted state. Some embodiments are based on the discovery that administering solid dosage form without food results in a higher total daily exposure (as measured by mean AUC₀₋₂₄) and/or maximum concentration (Cmax).

In some embodiments, consumption of food is permissible after about at least about 1 hour, at least about 2 hours, at least about 3 hours, at least about 4 hours following administration of the solid dosage form comprising leuprolide or the pharmaceutically acceptable salt thereof.

In some embodiments, the solid dosage form is administered once daily in the morning following an overnight fast. In some embodiments, the administration to the patient can occur for example, at least about 8 hours, at least about 9 hours, at least about 10 hours, at least about 11 hours or at least about 12 hours, or more after consuming food.

In some embodiments, the solid dosage form is administered twice daily at about a 10 hours, 11 hours, 12 hours, 13 hours or 14 hours interval. In some embodiments, consumption of food is permissible after about at least about 1 hour, at least about 2 hours, at least about 3 hours, at least about 4 hours following oral administration of leuprolide or the pharmaceutically acceptable salt thereof. In some embodiments, the second dose is administered up to 4 hours, up to 5 hours, up to 6 hours, after consuming food.

In some embodiments, half of the total daily dose is administered twice daily. In some embodiments, a first half and a second half of the daily dose are administered at about 10 hours to about 14 hours interval. In some embodiments, the first half of the daily dose is administered in the morning before consumption of food. In some embodiments, the second half of the daily dose is administered up to about 6 hours after consumption of food.

In some embodiments, the administration of the solid dosage form for a suitable period can reduce peak [GnRH- or GnRH agonist-] stimulated blood serum LH or 60 minutes following GnRH agonist stimulation to a concentration of <4.0 mIU/mL.

In some embodiments, the administration of the solid dosage form for a suitable period can reduce peak [GnRH- or GnRH agonist-] stimulated blood serum FSH or 60 minutes following GnRH agonist stimulation to a concentration of <2.5 mIU/mL.

In some embodiments, the administration of the solid dosage form for a suitable period can reduce the peak estradiol (in girls) to <20 pg/mL 30-40 minutes following GnRH stimulation or 60 minutes following GnRH agonist stimulation.

In some embodiments, the administration of the solid dosage form for a suitable period can reduce the peak testosterone (in boys) to <28.4 ng/dL following GnRH stimulation or 60 minutes following GnRH agonist stimulation.

In some embodiments, the administration of the solid dosage form for a suitable period can reduce basal blood serum estradiol in a female pediatric patient to a concentration of <5-10 pg/mL.

In some embodiments, the administration of the solid dosage form for a suitable period can reduce basal blood serum testosterone in a male pediatric patient to a concentration of <10-30 ng/dL, for example <25.3 ng/dL.

In some embodiments, the suitable period is 6 months to 36 months treatment period. In some embodiments, the suitable period is 12 months to 36 months treatment period. In some embodiments, the suitable period is until the pediatric patient reaches an appropriate age for puberty.

In some embodiments, the administration of the solid dosage form results in effective treatment of CPP in a pediatric patient. In some embodiments, the administration of the solid dosage form results in a decrease in the functional activity of the gonads (i.e. testes or ovaries), which may further result in decreased production or release of sex hormones. In some embodiments, the administration of the solid dosage form results to a return to a normal pre-pubertal state (e.g. suppressed LH and FSH, and subsequently estradiol and testosterone) for a pediatric patient.

In some embodiments, the administration of the solid dosage form results in reduction of blood serum concentrations of CPP-associated hormones, such as LH and FSH. In some embodiments, administration of the solid dosage form results in a suppression of LH to <0.6 mIU/mL and/or suppression of FSH to <0.5 mIU/mL.

In some embodiments, the administration of the solid dosage form can arrest or slow advancing skeletal bone growth measured as “skeletal bone age” following 6-12 months of treatment, and persisting over 5 years of treatment.

In some embodiments, the administration of the solid dosage form for a suitable period can reduce the mean bone growth velocity in a pediatric patient with CPP by about 10%, 20%, 25%, 30%, 35%, 40%, 45%. In some embodiments, the suitable period of time is about a twelve month treatment period or more.

In some embodiments, the administration of the solid dosage form for a suitable period can reduce the pediatric patient's mean ratio of bone age to chronological age at the time of measurement to from about 1.4 to about 1. In some embodiments, the suitable period of time is about a twelve month treatment period or more. For example, the administration of the solid dosage form can reduce the pediatric patient's mean ratio of bone age to chronological age at the time of measurement to about 1.4 over about a twelve month treatment period or the administration of the solid dosage form can reduce the pediatric patient's mean ratio of bone age to chronological age at the time of measurement to about 1.1 over about a sixty month treatment period.

In some embodiments, the administration of the solid dosage form for a suitable period can reduce the pediatric patient's mean ratio of bone age to chronological age at the time of measurement by about 5-21%. In some embodiments, the administration of the solid dosage form for a suitable period can reduce the pediatric patient's mean ratio of bone age to chronological age at the time of measurement by about 5% to about 15% over about a twelve month treatment period. In some embodiments, the administration of the solid dosage form for a suitable period can reduce the pediatric patient's mean ratio of bone age to chronological age at the time of measurement by about 20% or more over about a sixty month treatment period.

In some embodiments, the pediatric patient with CPP is treated with the solid dosage form for a time period of about 6 months, of about 12 months, of about 18 months, of about 24 months, of about 36 months, of about 48 months, of about sixty months or longer. In some embodiments, the pediatric patient with CPP is treated with the solid dosage form until the mean bone age substantially corresponds to the chronological age.

In some embodiments, the method of treatment of CPP provided herein reduces the development, advancement, or severity of at least one or more secondary sexual characteristics. In some instances, the secondary sexual characteristic may include, but are not limited to, bone growth velocity, bone growth age, Tanner stage, pubic har development, breast development, height, or weight. In some embodiments, the method of treatment of CPP provided herein results in the slowing or arrest of advancing breast and/or pubic hair development (Tanner Stages I-V) following 6-12 months of treatment, and persisting over 5 years of treatment.

In some embodiments, administration of the solid dosage form reduces the bone growth velocity of the pediatric patient from about 10% to about 25%, or from about 10% to about 50%, over about a 12-month period. In some embodiments, administration of the solid dosage form reduces the mean bone growth velocity of a pediatric patient to about 9 cm to 5 cm or less per year.

In some embodiments, the administration of the solid dosage form can reduce the mean bone growth velocity in a pediatric patient with CPP by about 10%, 20%, 25%, 30%, 35%, 40%, 45% over about a twelve-month treatment period. In some embodiments, the administration of the solid dosage form can reduce the pediatric patient's mean ratio of bone age to chronological age at the time of measurement to from about 1.4 to 1.3 over about a twelve month treatment period or more. In some embodiments, the administration of the solid dosage form can reduce the pediatric patient's mean ratio of bone age to chronological age at the time of measurement to from about 1.4 to about 1 over about a sixty month treatment period.

In some embodiments, the administration of the solid dosage form for a suitable period can reduce the pediatric patient's mean ratio of bone age to chronological age at the time of measurement by about 7%, 8%, 9%, 10%, 11%, 12%, 13%, 14%, 15%, 16% or 17% at about twelve-month treatment period.

In some embodiments, the pediatric patient with CPP is treated with the solid dosage form for a time period of about 6 months, of about 12 months, of about 18 months, of about 24 months, of about 36 months, of about 48 months, of about sixty or longer. In some embodiments, the pediatric patient with CPP is treated with the solid dosage form until the mean bone age substantially corresponds to the chronological age of the patient. In some embodiments, the pediatric patient with CPP is treated with the solid dosage form until the pediatric patient reaches an appropriate age for puberty.

In some embodiments, leuprolide (e.g. leuprolide acetate) can be co-administered with one or more active agent. In some embodiments, the one or more active agent is a synthetic progestin, estradiol or combination thereof. In some embodiments, the one or more active agent mitigates side effects of GnRH agonist therapy. Such side effects, include but are not limited to vasomotor symptoms (hot flashes) and accelerated bone demineralization. Vasomotor symptoms can be distressing, particularly in adults, interfering with comfort, and interrupting sleep. Bone demineralization can predispose over time to increased risk for osteoporosis and fractures, most typically lower back vertebrae and femoral neck (hip).

According to aspects illustrated herein, a pediatric patient having precocious puberty can be treated with a solid oral dosage form of the present disclosure. In some embodiments, such treatment includes orally administering the patient having precocious puberty a therapeutically effective amount of gonadotrophin releasing hormone (GnRH) analogue such as leuprolide, for a suitable period of time, a solid dosage form comprising (a) a therapeutically effective amount of gonadotrophin releasing hormone (GnRH) analogue; (b) an absorption enhancer; (c) coated acid particles intermixed with the GnRH analogue, wherein the coating separates the citric acid from the GnRH analogue in the tablet; (d) an outer layer of an acid-resistant enteric coating effective to transport the solid dosage form through the stomach of a patient while preventing contact between the GnRH analogue and stomach proteases; and (e) a water soluble barrier layer beneath the outer layer of enteric coating that separates the enteric coating from the coated acid particles, wherein the therapeutically effective amount of the gonadotrophin releasing hormone (GnRH) analogue amounts is between 40 to 120 mg per day (or any value there between as provided herein). In some embodiments, the solid dosage form can be administered once, twice or more daily. In some embodiments, the solid dosage from is a tablet.

In some embodiments, the solid dosage form may be administered orally in an effective amount within the dosage ranges described herein in a regimen of single or multiple (twice, etc.) daily or single or multiple weekly doses. In some embodiments, the solid dosage form may be administered orally in an effective amount for a suitable period of time to treat a disease in a subject in need thereof. As used herein, the term “a suitable period of time” refers to the period of time starting when a subject begins treatment for a condition using a method of the present disclosure, throughout the treatment, and up until when the subject stops treatment due to either a reduction in symptoms associated with the condition or due to a laboratory diagnosis indicating that the condition is under control. In an embodiment, a suitable period of time is from one (1) month and twelve (12) months or from one (1) month and twenty-four (24) months or more.

### Solid Dosage Form

In some embodiments, the solid dosage form comprises (a) a therapeutically effective amount of gonadotrophin releasing hormone (GnRH) analogue; (b) an absorption enhancer; (c) a pH lowering agent such as coated acid particles intermixed with the GnRH analogue, wherein the coating separates the citric acid from the GnRH analogue in the tablet; (d) an outer layer of an acid-resistant enteric coating effective to transport the solid dosage form through the stomach of a patient while preventing contact between the GnRH analogue and stomach proteases.

In some embodiments, degradation of the API by protease is suppressed by several mechanisms that would otherwise tend to cleave one or more of the peptide bonds of the active ingredient. In addition to natural amino acids, the amino acids may be D-amino acids or unnatural amino acids, some examples of which are discussed infra. The molecular structure may further include other substituents or modifications. Some peptides may be amidated at locations that are not amidated in nature, or may be otherwise modified.

In some embodiments, the solid dosage from comprises from about 0.01% (w/w) to about 20.0% (w/w) by weight of leuprolide relative to the total weight of the overall solid oral dosage form (exclusive of any acid-resistant protective coating). In some embodiments, about 0.01% (w/w) to about 15.0% (w/w) of leuprolide is present in the solid oral dosage form (exclusive of any acid-resistant protective coating). In some embodiments, about 0.01% (w/w) to about 10.0% (w/w) of leuprolide is present in the solid oral dosage form (exclusive of any acid-resistant protective coating). In some embodiments, about 0.01% (w/w) to about 1.0% (w/w) of leuprolide is present in the solid oral dosage form (exclusive of any acid-resistant protective coating). In some embodiments, about 0.01% (w/w) to about 0.1% (w/w) of leuprolide is present in the solid oral dosage form (exclusive of any acid-resistant protective coating). In some embodiments, about 0.1% (w/w) to about 20.0% (w/w) of leuprolide is present in the solid oral dosage form (exclusive of any acid-resistant protective coating). In some embodiments, about 0.1% (w/w) to about 15.0% (w/w) of leuprolide is present in the solid oral dosage form (exclusive of any acid-resistant protective coating).). In some embodiments, about 0.1% (w/w) to about 10.0% (w/w) of leuprolide is present in the solid oral dosage form (exclusive of any acid-resistant protective coating). In some embodiments, about 0.1% (w/w) to about 1.0% (w/w) of leuprolide is present in the solid oral dosage form (exclusive of any acid-resistant protective coating). In some embodiments, about 1.0% (w/w) to about 20.0% (w/w) of leuprolide is present in the solid oral dosage form (exclusive of any acid-resistant protective coating). In some embodiments, about 1.0% (w/w) to about 15.0% (w/w) of leuprolide is present in the solid oral dosage form (exclusive of any acid-resistant protective coating). In some embodiments, about 1.0% (w/w) to about 10.0% (w/w) of leuprolide is present in the solid oral dosage form (exclusive of any acid-resistant protective coating). In some embodiments, about 10.0% (w/w) to about 20.0% (w/w) of leuprolide is present in the solid oral dosage form (exclusive of any acid-resistant protective coating). In some embodiments, about 15.0% (w/w) to about 20.0% (w/w) of leuprolide is present in the solid oral dosage form (exclusive of any acid-resistant protective coating).

The pH-Lowering Agent (Acid) Proteolytic enzymes of both the stomach and intestines may degrade peptides, rendering them inactive before they can be absorbed into the bloodstream. Any amount of peptide that survives proteolytic degradation by proteases of the stomach (typically having acidic pH optima) is later confronted with proteases of the small intestine and enzymes secreted by the pancreas (typically having neutral to basic pH optima). Proteolytic degradation of peptides may contribute to limited systemic bioavailability of the peptide.

The acid is believed to lower the local intestinal pH (where the active agent has been released) to levels below the optimal range for many intestinal proteases. It is believed that this decrease in pH reduces the proteolytic activity of the intestinal proteases, thus affording protection to the peptide from potential degradation. The activity of these proteases is diminished by the temporarily acidic environment provided by the disclosure.

The total amount of the pH-lowering agent to be administered with each administration of peptide active ingredient should preferably be an amount which, when it is released into the intestine, is sufficient to lower the local intestinal pH substantially below the pH optima for proteases found there. The quantity required will necessarily vary with several factors including the type of pH-lowering agent used and the equivalents of protons provided by a given pH-lowering agent. In practice, the amount of pH-lowering agent expected to provide good bioavailability is an amount which, if the pharmaceutical composition of the disclosure were added to a solution of 10 milliliters of 0.1 M sodium bicarbonate, would lower the pH of that sodium bicarbonate solution to no higher than 5.5, and preferably no higher than 4.7, most preferably no higher than 3.5. The foregoing test for sufficient acidity is referenced elsewhere herein as “sodium bicarbonate test” and assumes sufficient passage of time for substantially complete dissolution of the pharmaceutical composition and intermixing thereof with the sodium bicarbonate solution.

The acid can for example promote neutralization and the collapse of the GI tract mucus layers, thereby enhancing absorption.

In some embodiments, the amount of pH-lowering agent used in a solid oral dosage form of the present disclosure ranges from about 50 milligrams up to about 500 milligrams. In some embodiments, the amount of pH-lowering agent used in a solid oral dosage form of the present disclosure ranges from about 75 milligrams up to about 500 milligrams. In some embodiments, the amount of pH-lowering agent used in a solid oral dosage form of the present disclosure ranges from about 100 milligrams up to about 500 milligrams. In some embodiments, the amount of pH-lowering agent used in a solid oral dosage form of the present disclosure ranges from about 150 milligrams up to about 500 milligrams. In some embodiments, the amount of pH-lowering agent used in a solid oral dosage form of the present disclosure ranges from about 200 milligrams up to about 500 milligrams. In some embodiments, the amount of pH-lowering agent used in a solid oral dosage form of the present disclosure ranges from about 250 milligrams up to about 500 milligrams. In some embodiments, the amount of pH-lowering agent used in a solid oral dosage form of the present disclosure ranges from about 300 milligrams up to about 500 milligrams. In some embodiments, the amount of pH-lowering agent used in a solid oral dosage form of the present disclosure ranges from about 350 milligrams up to about 500 milligrams. In some embodiments, the amount of pH-lowering agent used in a solid oral dosage form of the present disclosure ranges from about 400 milligrams up to about 500 milligrams. In some embodiments, the amount of pH-lowering agent used in a solid oral dosage form of the present disclosure ranges from about 450 milligrams up to about 500 milligrams. In some embodiments, the amount of pH-lowering agent used in a solid oral dosage form of the present disclosure ranges from about 50 milligrams up to about 450 milligrams. In some embodiments, the amount of pH-lowering agent used in a solid oral dosage form of the present disclosure ranges from about 100 milligrams up to about 450 milligrams. In some embodiments, the amount of pH-lowering agent used in a solid oral dosage form of the present disclosure ranges from about 150 milligrams up to about 450 milligrams. In some embodiments, the amount of pH-lowering agent used in a solid oral dosage form of the present disclosure ranges from about 200 milligrams up to about 450 milligrams. In some embodiments, the amount of pH-lowering agent used in a solid oral dosage form of the present disclosure ranges from about 250 milligrams up to about 450 milligrams. In some embodiments, the amount of pH-lowering agent used in a solid oral dosage form of the present disclosure ranges from about 300 milligrams up to about 450 milligrams. In some embodiments, the amount of pH-lowering agent used in a solid oral dosage form of the present disclosure ranges from about 350 milligrams up to about 450 milligrams. In some embodiments, the amount of pH-lowering agent used in a solid oral dosage form of the present disclosure ranges from about 400 milligrams up to about 450 milligrams. In some embodiments, the amount of pH-lowering agent used in a solid oral dosage form of the present disclosure ranges from about 50 milligrams up to about 400 milligrams. In some embodiments, the amount of pH-lowering agent used in a solid oral dosage form of the present disclosure ranges from about 100 milligrams up to about 400 milligrams. In some embodiments, the amount of pH-lowering agent used in a solid oral dosage form of the present disclosure ranges from about 150 milligrams up to about 400 milligrams. In some embodiments, the amount of pH-lowering agent used in a solid oral dosage form of the present disclosure ranges from about 200 milligrams up to about 400 milligrams. In some embodiments, the amount of pH-lowering agent used in a solid oral dosage form of the present disclosure ranges from about 250 milligrams up to about 400 milligrams. In some embodiments, the amount of pH-lowering agent used in a solid oral dosage form of the present disclosure ranges from about 300 milligrams up to about 400 milligrams. In some embodiments, the amount of pH-lowering agent used in a solid oral dosage form of the present disclosure ranges from about 350 milligrams up to about 400 milligrams. In some embodiments, the amount of pH-lowering agent used in a solid oral dosage form of the present disclosure ranges from about 50 milligrams up to about 350 milligrams. In some embodiments, the amount of pH-lowering agent used in a solid oral dosage form of the present disclosure ranges from about 100 milligrams up to about 350 milligrams. In some embodiments, the amount of pH-lowering agent used in a solid oral dosage form of the present disclosure ranges from about 150 milligrams up to about 350 milligrams. In some embodiments, the amount of pH-lowering agent used in a solid oral dosage form of the present disclosure ranges from about 200 milligrams up to about 350 milligrams. In some embodiments, the amount of pH-lowering agent used in a solid oral dosage form of the present disclosure ranges from about 250 milligrams up to about 350 milligrams. In some embodiments, the amount of pH-lowering agent used in a solid oral dosage form of the present disclosure ranges from about 300 milligrams up to about 350 milligrams. In some embodiments, the amount of pH-lowering agent used in a solid oral dosage form of the present disclosure ranges from about 50 milligrams up to about 300 milligrams. In some embodiments, the amount of pH-lowering agent used in a solid oral dosage form of the present disclosure ranges from about 100 milligrams up to about 300 milligrams. In some embodiments, the amount of pH-lowering agent used in a solid oral dosage form of the present disclosure ranges from about 150 milligrams up to about 300 milligrams. In some embodiments, the amount of pH-lowering agent used in a solid oral dosage form of the present disclosure ranges from about 200 milligrams up to about 300 milligrams. In some embodiments, the amount of pH-lowering agent used in a solid oral dosage form of the present disclosure ranges from about 250 milligrams up to about 300 milligrams. In some embodiments, the amount of pH-lowering agent used in a solid oral dosage form of the present disclosure ranges from about 50 milligrams up to about 250 milligrams. In some embodiments, the amount of pH-lowering agent used in a solid oral dosage form of the present disclosure ranges from about 100 milligrams up to about 250 milligrams. In some embodiments, the amount of pH-lowering agent used in a solid oral dosage form of the present disclosure ranges from about 150 milligrams up to about 250 milligrams. In some embodiments, the amount of pH-lowering agent used in a solid oral dosage form of the present disclosure ranges from about 200 milligrams up to about 250 milligrams. In some embodiments, the amount of pH-lowering agent used in a solid oral dosage form of the present disclosure ranges from about 50 milligrams up to about 200 milligrams. In some embodiments, the amount of pH-lowering agent used in a solid oral dosage form of the present disclosure ranges from about 100 milligrams up to about 200 milligrams. In some embodiments, the amount of pH-lowering agent used in a solid oral dosage form of the present disclosure ranges from about 150 milligrams up to about 200 milligrams. In some embodiments, the amount of pH-lowering agent used in a solid oral dosage form of the present disclosure ranges from about 50 milligrams up to about 150 milligrams. In some embodiments, the amount of pH-lowering agent used in a solid oral dosage form of the present disclosure ranges from about 100 milligrams up to about 150 milligrams. In some embodiments, the amount of pH-lowering agent used in a solid oral dosage form of the present disclosure ranges from about 100 milligrams up to about 100 milligrams. In some embodiments, the amount of pH-lowering agent used in a solid oral dosage form of the present disclosure is about 50 milligrams. In some embodiments, the amount of pH-lowering agent used in a solid oral dosage form of the present disclosure is about 75 milligrams. In some embodiments, the amount of pH-lowering agent used in a solid oral dosage form of the present disclosure is about 100 milligrams. In some embodiments, the amount of pH-lowering agent used in a solid oral dosage form of the present disclosure is about 125 milligrams. In some embodiments, the amount of pH-lowering agent used in a solid oral dosage form of the present disclosure is about 150 milligrams. In some embodiments, the amount of pH-lowering agent used in a solid oral dosage form of the present disclosure is about 175 milligrams. In some embodiments, the amount of pH-lowering agent used in a solid oral dosage form of the present disclosure is about 200 milligrams. In some embodiments, the amount of pH-lowering agent used in a solid oral dosage form of the present disclosure is about 225 milligrams. In some embodiments, the amount of pH-lowering agent used in a solid oral dosage form of the present disclosure is about 250 milligrams. In some embodiments, the amount of pH-lowering agent used in a solid oral dosage form of the present disclosure is about 300 milligrams. In some embodiments, the amount of pH-lowering agent used in a solid oral dosage form of the present disclosure is about 325 milligrams. In some embodiments, the amount of pH-lowering agent used in a solid oral dosage form of the present disclosure is about 350 milligrams. In some embodiments, the amount of pH-lowering agent used in a solid oral dosage form of the present disclosure is about 375 milligrams. In some embodiments, the amount of pH-lowering agent used in a solid oral dosage form of the present disclosure is about 400 milligrams. In some embodiments, the amount of pH-lowering agent used in a solid oral dosage form of the present disclosure is about 425 milligrams. In some embodiments, the amount of pH-lowering agent used in a solid oral dosage form of the present disclosure is about 450 milligrams. In some embodiments, the amount of pH-lowering agent used in a solid oral dosage form of the present disclosure is about 475 milligrams. In some embodiments, the amount of pH-lowering agent used in a solid oral dosage form of the present disclosure is about 500 milligrams or higher. The foregoing preferences relate to the total combined weight of all pH-lowering agents where two or more of such agents are used in combination.

The pH-lowering agent may be any pharmaceutically acceptable compound that is not toxic in the gastrointestinal tract and is capable of either delivering hydrogen ions (a traditional acid) or of inducing higher hydrogen ion content from the local environment. It may also be any combination of such compounds. In some embodiments, the at least one pH-lowering agent has a pKa no higher than 4.2, or no higher than 3.0. In some embodiments, the pH lowering agent has a solubility in water of at least 30 grams per 100 milliliters of water at room temperature. In some embodiments, organic acids are used.

Examples of compounds that induce higher hydrogen ion content include aluminum chloride and zinc chloride. Pharmaceutically acceptable traditional acids include, but are not limited to acid salts of amino acids (e.g. amino acid hydrochlorides) or derivatives thereof. Examples of these are acid salts of acetylglutamic acid, alanine, arginine, asparagine, aspartic acid, betaine, camitine, camosine, citrulline, creatine, glutamic acid, glycine, histidine, hydroxylysine, hydroxyproline, hypotaurine, isoleucine, leucine, lysine, methylhistidine, norleucine, ornithine, phenylalanine, proline, sarcosine, serine, taurine, threonine, tryptophan, tyrosine and valine.

Other examples of useful pH-lowering compounds include carboxylic acids such as acetylsalicylic, acetic, ascorbic, citric, fumaric, glucuronic, glutaric, glyceric, glycocolic, glyoxylic, isocitric, isovaleric, lactic, maleic, oxaloacetic, oxalosuccinic, propionic, pyruvic, succinic, tartaric, valeric, and the like.

Other useful pH-lowering agents that might not usually be called “acids” in the art, but which may nonetheless be useful in accordance with the disclosure are phosphate esters (e.g., fructose 1, 6 diphosphate, glucose 1, 6 diphosphate, phosphoglyceric acid, and diphosphoglyceric acid). CARBOPOL® (Trademark BF Goodrich) and polymers such as polycarbophil may also be used to lower pH.

Any combination of pH lowering agents that achieves the required pH level of no higher than 5.5 in the sodium bicarbonate test discussed supra may be used. Some embodiments utilize, as at least one of the pH-lowering agents of the pharmaceutical composition, an acid selected from the group consisting of citric acid, tartaric acid and an acid salt of an amino acid.

In some embodiments, regardless of the acid chosen, the acid is in the form of acid particles coated with a protective coating.

In some embodiments, the weight ratio of pH-lowering agent to the API can be from 0.1:1 to 10,000:1. In some embodiments, the weight ratio of pH-lowering agent to the API can exceed 4:1, 20:1, 200:1, or 800:1 or 2000:1.

In some embodiments, the acid is provided, at least in part, by acid particles coated with a protective coating to reduce undesirable acid interaction with other components of the formulation, such as the peptide active agent and, where used, the outer enteric coating. In some embodiments, the coating is an hydrophilic coating. In some embodiments, the coating is water soluble coating.

When coated acid particles are used, the particles are coated with a pharmaceutically acceptable protective coating that is non-acidic and preferably has a solubility in water of at least one gram, and preferably at least 10 grams, per 100 milliliters of water at room temperature. As the coating is for the purpose of reducing acid interaction with other components of the pharmaceutical composition, it is important that the coating not itself be acidic such that its own acidity could undesirably cause some of the acid interactions that it is the coating's purpose to prevent.

Appropriate coating materials include but are not limited to monosaccharides (e.g. glucose and fructose), polysaccharides (e.g. maltodextrin), and acid salts (e.g. sodium citrate). When acid salts are used, it is preferred, but not required, that they be salts of the acid being coated (e.g., sodium citrate-coated citric acid particles). In some embodiments, coated citric acid particles used in a solid dosage form of the present disclosure are Citric Acid DC available from Jungbunzlauer. Citric Acid DC is a direct compressible type of citric acid coated with a thin layer of maltodextrin. In some embodiments, coated citric acid particles used in a solid dosage form of the present disclosure are CITROCOAT® N available from Jungbunzlauer. CITROCOAT® N is comprised of citric acid as the core material, with a layer of a citrate as shell material. When used as the acid, citric acid or other organic acids can be coated by spraying a coating solution which contains, for example, glucose or sodium citrate onto granules of an organic acid in a fluid-bed dryer. Coatings discussed herein may be used on particles of other acids discussed herein.

In some embodiments, the average size of the acid-coated particles is from 30 mesh to 140 mesh.

### Water Soluble Barrier Layer

The water soluble barrier layer can be comprised of a compound that is water soluble in both acidic and basic environments. Examples of compounds useful for this purpose include but are not limited to hydroxypropylmethylcellulose, hydroxypropylcellulose, methylcellulose, polyvinylpyrrolidone, and polyvinyl alcohol/polyethylene glycol graft copolymer. Preferably, water solubility is at least one gram, more preferably at least 11 grams, per 100 milliliters at room temperature. Polyvinyl alcohol/polyethylene glycol graft copolymer is preferred in some embodiments. In some embodiments water solubility, at both pH 6.0 and pH 8.0, is in excess of 12 grams per 100 milliliters of water at room temperature. Good solubility in both acid and basic pH aids desirable quick dissolution in the intestinal region where pH is generally basic, but where the pharmaceutical composition's release of significant quantities of acid might at least temporarily impede dissolution of a material that was not also readily soluble in an aqueous acid environment. In some embodiments, the water-soluble barrier layer is present at a weight which is from about 7.0% to about 12.0% of the weight of the remainder of the solid oral dosage form. In some embodiments, the water-soluble barrier layer is present at a weight of about 7.0% of the weight of the remainder of the solid oral dosage form. In some embodiments, the water-soluble barrier layer is present at a weight of about 12.0% of the weight of the remainder of the solid oral dosage form. In some embodiments, the water-soluble barrier layer is present at a weight of from about 6.0% to about 15.0% of the weight of the remainder of the solid oral dosage form. In some embodiments, the water-soluble barrier layer is present at a weight of about 13.0% to about 15.0% of the weight of the remainder of the solid oral dosage form. In some embodiments, the water-soluble barrier layer is present at a weight of from about 12 to about 20 mg/cm².

### Absorption/Permeation Enhancer

In some embodiments, an absorption enhancer (also referred herein as permeation enhancer) is included in the solid oral dosage form. The absorption enhancers can be present in a quantity that constitutes from 0.1 to 20.0 percent by weight, relative to the overall weight of the solid oral dosage form (exclusive of any enteric coating).

In some embodiments, the solid dosage form comprises from about 50 mg to about 300 mg of absorption enhancers. In some embodiments, the solid dosage form comprises from about 100 mg to about 300 mg of absorption enhancers. In some embodiments, the solid dosage form comprises from about 150 mg to about 300 mg of absorption enhancers. In some embodiments, the solid dosage form comprises from about 200 mg to about 300 mg of absorption enhancers. In some embodiments, the solid dosage form comprises from about 250 mg to about 300 mg of absorption enhancers. In some embodiments, the solid dosage form comprises from about 50 mg to about 100 mg of absorption enhancers. In some embodiments, the solid dosage form comprises from about 50 mg to about 150 mg of absorption enhancers. In some embodiments, the solid dosage form comprises from about 50 mg to about 200 mg of absorption enhancers. In some embodiments, the solid dosage form comprises from about 50 mg to about 250 mg of absorption enhancers. In some embodiments, the solid dosage form comprises from about 100 mg to about 150 mg of absorption enhancers. In some embodiments, the solid dosage form comprises from about 100 mg to about 200 mg of absorption enhancers. In some embodiments, the solid dosage form comprises from about 100 mg to about 250 mg of absorption enhancers. In some embodiments, the solid dosage form comprises from about 150 mg to about 200 mg of absorption enhancers. In some embodiments, the solid dosage form comprises from about 150 mg to about 250 mg of absorption enhancers. In some embodiments, the solid dosage form comprises from about 200 mg to about 250 mg of absorption enhancers. In some embodiments, the solid dosage form comprises about 50 mg of absorption enhancers. In some embodiments, the solid dosage form comprises about 100 mg of absorption enhancers. In some embodiments, the solid dosage form comprises about 150 mg of absorption enhancers. In some embodiments, the solid dosage form comprises about 200 mg of absorption enhancers. In some embodiments, the solid dosage form comprises about 250 mg of absorption enhancers. In some embodiments, the solid dosage form comprises about 300 mg of absorption enhancers.

In some embodiments, the solid dosage form comprises from about 50 mg to about 300 mg of lauroyl-L-carnitine. In some embodiments, the solid dosage form comprises from about 100 mg to about 300 mg of lauroyl-L-camitine. In some embodiments, the solid dosage form comprises from about 150 mg to about 300 mg of lauroyl-L-camitine. In some embodiments, the solid dosage form comprises from about 200 mg to about 300 mg of lauroyl-L-carnitine. In some embodiments, the solid dosage form comprises from about 250 mg to about 300 mg of lauroyl-L-camitine. In some embodiments, the solid dosage form comprises from about 50 mg to about 100 mg of lauroyl-L-carnitine. In some embodiments, the solid dosage form comprises from about 50 mg to about 150 mg of lauroyl-L-carnitine. In some embodiments, the solid dosage form comprises from about 50 mg to about 200 mg of lauroyl-L-carnitine. In some embodiments, the solid dosage form comprises from about 50 mg to about 250 mg of lauroyl-L-carnitine. In some embodiments, the solid dosage form comprises from about 100 mg to about 150 mg of lauroyl-L-carnitine. In some embodiments, the solid dosage form comprises from about 100 mg to about 200 mg of lauroyl-L-carnitine. In some embodiments, the solid dosage form comprises from about 100 mg to about 250 mg of lauroyl-L-carnitine. In some embodiments, the solid dosage form comprises from about 150 mg to about 200 mg of lauroyl-L-carnitine. In some embodiments, the solid dosage form comprises from about 150 mg to about 250 mg of lauroyl-L-carnitine. In some embodiments, the solid dosage form comprises from about 200 mg to about 250 mg of lauroyl-L-carnitine. In some embodiments, the solid dosage form comprises about 50 mg of lauroyl-L-carnitine. In some embodiments, the solid dosage form comprises about 100 mg of lauroyl-L-carnitine. In some embodiments, the solid dosage form comprises about 150 mg of lauroyl-L-carnitine. In some embodiments, the solid dosage form comprises about 200 mg of lauroyl-L-carnitine. In some embodiments, the solid dosage form comprises about 250 mg of lauroyl-L-carnitine. In some embodiments, the solid dosage form comprises about 300 mg of lauroyl-L-carnitine.

In some embodiments, absorption enhancers can be surface active agents which act both as solubility enhancers and uptake enhancers. Generically speaking, “solubility enhancers” improve the ability of the components of the disclosure to be solubilized in either the aqueous environment into which they are originally released or into the lipophilic environment of the mucous layer lining the intestinal walls, or both. “Transport (uptake) enhancers” (which are frequently the same surface active agents used as solubility enhancers) are those which facilitate the ease by which API cross the intestinal wall.

One or more absorption enhancers may perform one function only (e.g., solubility), or one or more absorption enhancers may perform the other function only (e.g., uptake), within the scope of the disclosure. It is also possible to have a mixture of several compounds some of which provide improved solubility, some of which provide improved uptake and/or some of which perform both functions. Without intending to be bound by theory, it is believed that uptake enhancers may act by (1) increasing disorder of the hydrophobic region of the membrane exterior of intestinal cells, allowing for increased transcellular transport; or (2) leaching membrane proteins resulting in increased transcellular transport; or (3) widening the pore radius between cells for increased paracellular transport.

Surface active agents are believed to be useful both as solubility enhancers and as uptake enhancers. For example, detergents are useful in (1) solubilizing all of the active components quickly into the aqueous environment where they are originally released, (2) enhancing lipophilicity of the components of the disclosure, especially the API, aiding its passage into and through the intestinal mucus, (3) enhancing the ability of the normally polar API to cross the epithelial barrier of the brush border membrane; and (4) increasing transcellular and/or paracellular transport as described above.

In some embodiments, when surface active agents are used as the absorption enhancers, they can be free flowing powders for facilitating the mixing and loading of during the manufacturing process. Because of inherent characteristics of some peptide active ingredients (e.g., their isoelectric point, molecular weight, amino acid composition, etc.) certain surface active agents interact best with certain peptides. In some embodiments, the surface active agent used is as an absorption enhancer selected from the group consisting of (i) anionic surface active agents, (ii) cationic surface agents, (iii) non-ionic surface active agents, and (iv) mixtures of anionic surface active agents (especially those having linear hydrocarbon regions) together with negative charge neutralizers. Negative charge neutralizers include but are not limited to acyl carnitines, cetyl pyridinium chloride, and the like. In some embodiments, the anionic surface active agent is one of a cholesterol derivative (e.g., bile acids), sodium lauryl sulfate (SLS, also known as Sodium dodecyl sulfate (SDS)) or a combination thereof. In some embodiments, the cationic surface agent is an acylcarnitine or the like.

In some embodiments, the absorption enhancer is soluble at acid pH, particularly in the 3.0 to 5.0 range.

In some embodiments, a mixture of cationic surface active agents and anionic surface active agents that are cholesterol derivatives, both of which are soluble at acid pH, are used.

In some embodiments, an acid soluble bile acid is used together with a cationic surface active agent. In some embodiments, an acyl carnitine and a sucrose ester is used. In some embodiments, when a particular absorption enhancer is used alone, it is a cationic surface active agent. Acyl camitines (e.g., lauroyl camitine), phospholipids and bile acids are particularly good absorption enhancers, especially acyl camitine. Anionic surfactants such as cholesterol derivatives or SLS are also used in some embodiments. Sodium lauryl sulfate is an hydrophobic surfactant. In some embodiments, the permeation enhancer is sodium dodecyl sulphate (SDS) or a pharmaceutically acceptable surfactant having suitable CMC.

It is the intent of these preferences to avoid interactions with the API that interfere with absorption of active ingredient into the blood. For example, the API may become entrapped in micelles of the surfactant, and thus physically unavailable for permeation across the intestinal epithelium.

To reduce the likelihood of side effects, preferred detergents, when used as the absorption enhancers of the disclosure, are either biodegradable or reabsorbable (e.g. biologically recyclable compounds such as bile acids, phospholipids, and/or acyl camitines), preferably biodegradable. Acyl carnitines are believed particularly useful in enhancing paracellular transport. When a bile acid (or another anionic detergent lacking linear hydrocarbons) is used in combination with a cationic detergent, some peptides, can be better transported both to and through the intestinal wall. In some embodiments, and without intending to be bound by theory, cationic ion exchange agents (e.g. detergents) are included to provide solubility enhancement by another possible mechanism. In particular, they may prevent the binding of the peptide active ingredient to mucus. Preferred cationic ion exchange agents include protamine chloride or any other polycation.

Preferred absorption enhancers include one or more of: (a) salicylates such as sodium salicylate, 3-methoxysalicylate, 5-methoxysalicylate and homovanilate; (b) bile acids such as taurocholic, tauorodeoxycholic, deoxycholic, cholic, glycholic, lithocholate, chenodeoxycholic, ursodeoxycholic, ursocholic, dehydrocholic, fusidic, etc.; (c) non-ionic surfactants such as polyoxyethylene ethers (e.g. Brij 36T, Brij 52, Brij 56, Brij 76, Brij 96, Texaphor A6, Texaphor A14, Texaphor A60 etc.), p-t-octyl phenol polyoxyethylenes (Triton X-45, Triton X-100, Triton X-114, Triton X-305 etc.) nonylphenoxypoloxyethylenes (e.g. Igepal CO series), polyoxyethylene sorbitan esters (e.g. Tween-20, Tween-80 etc.); (d) anionic surfactants such as dioctyl sodium sulfosuccinate; (e) lyso-phospholipids such as lysolecithin and lysophosphatidylethanolamine; (f) acylcarnitines, acylcholines and acyl amino acids such as lauroyl-L-carnitine, myristoylcamitine, palmitoylcarnitine, lauroylcholine, myristoylcholine, palmitoylcholine, hexadecyllysine, N-acylphenylalanine, N-acylglycine etc.; g) water soluble phospholipids such as diheptanoylphosphatidylcholine, dioctylphosphatidylcholine etc.; (h) medium-chain glycerides which are mixtures of mono-, di- and triglycerides containing medium-chain-length fatty acids (caprylic, capric and lauric acids); (i) ethylene-diaminetetraacetic acid; (j) cationic surfactants such as cetylpyridinium chloride; (k) fatty acid derivatives of polyethylene glycol such as Labrasol, Labrafac, etc.; (1) alkylsaccharides such as lauryl maltoside, lauroyl sucrose, myristoyl sucrose, palmitoyl sucrose, etc. . . . , (m) sodium lauryl sulfate (SLS), or a pharmaceutically acceptable surfactant having similar CMC or any combinations of the foregoing.

### Acid-Resistant Protective Vehicle

An acid-resistant protective vehicle can be utilized to separate the peptide compound from stomach proteases. Any carrier or vehicle that protects the peptide from stomach proteases and then dissolves so that the other ingredients of the disclosure may be released in the intestine is suitable. Examples include cellulose acetate phthalate, hydroxypropyl methylethylcellulose succinate, hydroxypropyl methylcellulose phthalate, carboxyl methylethylcellulose and methacrylic acid-methyl methacrylate copolymer. In some embodiments, the peptide, absorption enhancers such as solubility and/or uptake enhancer(s) (when included), chymotrypsin inhibitor, and pH-lowering agent(s), are included in a sufficiently viscous protective syrup to permit protected passage of the components of the disclosure through the stomach.

Suitable enteric coatings for protecting the peptide from stomach proteases may be applied, for example, to capsules after the remaining components of the disclosure have been loaded within the capsule. In other embodiments, enteric coating is coated on the outside of a tablet or coated on the outer surface of particles of active components which are then pressed into tablet form, or loaded into a capsule, which is itself preferably coated with an enteric coating.

It is desirable that all components of the disclosure be released from the carrier or vehicle, and solubilized in the intestinal environment as simultaneously as possible. In some embodiments, the vehicle or carrier can release the active components in the small intestine where uptake enhancers that increase transcellular or paracellular transport are less likely to cause undesirable side effects than if the same uptake enhancers were later released in the colon. It is emphasized, however, that the present disclosure is believed effective in the colon as well as in the small intestine. In some embodiments, the acid resistant protective vehicle is present at a weight which is from about 3.0% to about 10.0% of the weight of the remainder of the solid oral dosage form (the “remainder” being the solid oral dosage form exclusive of enteric coating itself). In some embodiments, the acid resistant protective vehicle is present at a weight of about 3.6% of the weight of the remainder of the solid oral dosage form. In some embodiments, the acid resistant protective vehicle is present at a weight of about 7.0% of the weight of the remainder of the solid oral dosage form. In some embodiments, the acid resistant protective vehicle is present at a weight of about 10.0% of the weight of the remainder of the solid oral dosage form. In some embodiments, the acid resistant protective vehicle is present at a weight of from about 5 to about 15 mg/cm². In some embodiments, the acid resistant protective vehicle is present at a weight of from about 5 to about 10 mg/cm². In some embodiments, the acid resistant protective vehicle is present at a weight of from about 10 to about 15 mg/cm². In some embodiments, the acid resistant protective vehicle is present at a weight of about 5, 6, 7, 8, 9, 10, 11, 12, 13, 14, or 15 mg/cm². In some embodiments, a composition of the present disclosure is an enteric coated capsule that is sufficient to prevent breakdown of the solid oral dosage form of the disclosure in 0.1N HCl for at least two hours, then capable of permitting complete release of all contents of the solid oral dosage form within thirty minutes after pH is increased to 6.8 or pH 6.8 to 7.5 in a dissolution bath in which said composition is rotating at greater than 100 revolutions per minute. In embodiments in which the water-soluble barrier layer of the disclosure is used, less enteric coating may be required, sometimes less than the amount of water-soluble barrier layer.

In some embodiments, a composition of the present disclosure is an enteric coated tablet that is sufficient to prevent breakdown of the solid oral dosage form of the disclosure in 0.1N HCl for at least two hours, then capable of permitting complete release of all contents of the solid oral dosage form within thirty-sixty minutes after pH is increased to 6.8 (e.g. pH 6.8-7.5) in an apparatus 2 (paddle) in which said composition is rotating at greater than 100 revolutions per minute.

### Filler

In some embodiments, the filler is a cellulose derivative filler, such as carboxymethylcellulose, cellulose acetate, hydroxyethylcellulose, hydroxypropylcellulose, hydroxypropylmethylcellulose. To achieve the desired release profile, a high molecular weight (high viscosity) hydroxypropylcellulose (HPC) can be used. High molecular weight (high viscosity) hydroxypropylcellulose is known to effectively sustain the release of drugs. In some embodiments, the filler has a viscosity of about 3,000 to 120,000 cP at 20° C.

In some embodiments, a filler such as a cellulose filler like PROSOLV™ available from JRS Pharma can be utilized. In some embodiments, a cellulose filler such as Avicel™ PH (microcrystalline cellulose) available from FMC BioPolymer may be utilized. In some embodiments, a cellulose filler such as Avicel™ HFE (microcrystalline cellulose, co-processed with mannitol) available from FMC BioPolymer may be utilized. In some embodiments, a filler such as, Pearlitol™ (mannitol) available from Roquette Freres may be utilized. Other fillers are known in the art.

### Optional Pharmaceutical Binder

In some embodiments, the solid oral dosage form is in tablet form and a pharmaceutical binder can be included in the solid oral dosage form. Preferred binders include but are not limited to KOLLIDON VA64, KOLLIDON VA64 fine, KOLLIDON 30, AVICEL PH-101, HPC, PHARMACOAT 606, and MALDEX.

### Optional Pharmaceutical Disintegrant

In some embodiments, a pharmaceutical tablet is used as a preferred single oral dosage form. In some embodiments, a pharmaceutically acceptable disintegrant is included. In some embodiments, a pharmaceutically acceptable super-disintegrant is included. Any disintegrant that performs the function of enhancing dissolution speed may be used. In some embodiments, the disintegrants include but are not limited to KOLLIDON CL, POLYPLASDONE, EXPLOTAB, and AC-DI-SOL, available from International Specialty Products, JRS Pharma and FMC Biopolymer, respectively. In some embodiments, the disintegrant is present in an amount between 1 and 15 percent by weight relative to the total tablet weight (% wt, when tablets are used), exclusive of any water-soluble barrier layer and any acid-resistant protective vehicle. In some embodiments, the solid oral dosage form may include a reduced amount of disintegrant. In some embodiments, the solid oral dosage comprises less than 4% wt, less than 3.5% wt, less than 3% wt, less than 2.5% wt, less than 2% wt, less than 1.5% wt, less than 1% wt, less than 0.5% wt, less than 0.01% wt, less than 0.001% wt disintegrant. In some embodiments, the solid oral dosage does not include a tablet disintegrant.

### Optional Pharmaceutical Glidant

In some embodiments, a pharmaceutically acceptable glidant is included. Any glidant that performs the function of enhancing powder flow may be used. Preferred glidants include but are not limited to talc, calcium silicate, magnesium silicate, silicon dioxide. Preferably, the glidant is present in an amount between 0.1 and 2.0 percent by weight relative to the weight of the pharmaceutical composition, exclusive of any water-soluble barrier layer and any acid-resistant protective vehicle.

### Optional Pharmaceutical Lubricant

In some embodiments, a pharmaceutically acceptable lubricant is included. Any lubricant that performs the function of preventing powder from sticking to the tooling may be used. Preferred lubricants include but are not limited to stearic acid, magnesium stearate, and hydrogenated vegetable oil type 1. In some embodiments, the lubricant is present in an amount between 0.2 and 5.0 percent by weight relative to the weight of the solid oral dosage form, exclusive of any water-soluble barrier layer and any acid-resistant protective vehicle.

### Optional Antioxidant

In some embodiments, a pharmaceutically acceptable antioxidant is included. Any antioxidant that performs the function of preventing the oxidation of labile amino acids in peptides, such as methionine or tryptophan may be used. Preferred antioxidants include but are not limited to sodium pyruvate, derivatives of sodium pyruvate, ascorbic acid, ascorbyl palmitate, butylated hydroxyanisole, butylated hydroxytoluene, sodium bisulfite, and sodium metabisulfite. In some embodiments, the antioxidant is present in an amount between 0.5 and 5 mg per tablet.

### Miscellaneous Other Optional Ingredients

In some embodiments, another peptide (such as albumin, casein, soy protein, other animal or vegetable proteins and the like) can be included to reduce non-specific adsorption (e.g., binding of peptide to the intestinal mucus barrier) thereby lowering the necessary concentration of the expensive peptide active ingredient. When added, the peptide is in some embodiments, from 1.0 to 10.0 percent by weight relative to the weight of the overall solid oral dosage form (excluding any water-soluble barrier layer and any acid-resistant protective vehicle). Preferably, this additional peptide is not physiologically active and is most preferably a food peptide such as soybean peptide or the like. Without intending to be bound by theory, this additional non-physiologically active peptide, may also increase bioavailability by acting as a protease scavenger that desirably competes with the peptide active ingredient for protease interaction. The second peptide may also aid the active compound's passage through the liver.

All solid oral dosage forms of the present disclosure may optionally also include common pharmaceutical carriers, diluents or fillers. The solid oral dosage forms may include gelatin capsules, preservatives, colorants and the like in their usual known sizes and amounts.

The optional ingredients discussed herein are not exclusive. Other pharmaceutically acceptable agents may also be included. All optional components may be combined in any combination. Because most preferences stated herein provide benefits by different mechanisms, such combinations should be beneficial.

### Inhibitor of Proteolytic Degradation of the Active Agent

When the active agent is a protein or a peptide, a protease inhibitor may be added that prevents or reduces the proteolytic degradation of the active agent, which may occur under in the environmental conditions of the gastrointestinal tract. Preferably, the protease inhibitor that prevents or reduces the proteolytic degradation of the active agent should be pharmaceutically acceptable in relation to the certain application in animals or in humans. In some embodiments, inhibitors of trypsin or chymotrypsin can be added.

When prepared in tablet form, it is preferred that the maximum weight loss during friability testing be no greater than 1%. As used herein, friability testing refers to the technique described in “Tablet Friability”, Chapter 1216, USP 28 page 2745.

When absorption enhancers are used, it is preferred that the weight ratio of pH-lowering agent(s) (exclusive of coating on any coated acid particles being used) to absorption enhancer(s) be between 5:2 and 20:1, 4:1-12:1, or between 5:1-10:1. The total weight of all pH-lowering agents and the total weight of all absorption enhancers in a given solid oral dosage forms is included in the foregoing preferred ratios. For example, if a solid oral dosage forms includes two pH-lowering agents and three absorption enhancers, the foregoing ratios will be computed on the total combined weight of both pH-lowering agents and the total combined weight of all three absorption enhancers.

In some embodiments, the pH-lowering agent, the API, the absorption enhancer, when used, (whether single compounds or a plurality of compounds in each category) are uniformly dispersed in the solid oral dosage forms. In some embodiments, the solid oral dosage forms comprise granules that include a pharmaceutical binder having the API, the pH-lowering agent and the absorption enhancer uniformly dispersed within said binder. In some embodiments, granules may consist of an acid core, surrounded by a uniform layer of organic acid, a layer of enhancer and a layer of the API that is surrounded by an outer layer of organic acid. Granules may be prepared from an aqueous mixture consisting of pharmaceutical binders such as polyvinyl pyrrolidone or hydroxypropyl methylcellulose, together with the pH-lowering agents, optional absorption enhancers, and API of the disclosure.

In some embodiments, API, acid (e.g. coated acid particles), absorption enhancer, a pharmaceutical binder (when necessary), a disintegrant (when necessary), a glidant, a stabilizer (when necessary) and a lubricant are thoroughly intermixed, compressed into tablet form, coated with a water-soluble barrier layer (preferably adding at least about 3% to the weight of the tablet (e.g. about 3-6%), which is in turn coated with an enteric coating that adds another about 4-15% to the weight of the tablet (e.g. 4-7%). In some embodiments, the water soluble layer adds more than the enteric coating (e.g. 6% and 4%, respectively).

In some embodiments, a single tablet is used at each administration and administration provides simultaneous release of the API, pH-lowering agent and absorption enhancers. This is desirable because the acid is best able to reduce undesirable proteolytic attack on the peptide when released in close time proximity to release of the peptide. Near simultaneous release is best achieved by administering all components of the disclosure as a single tablet. However, the disclosure also includes, for example, dividing the required amount of acid, and enhancers among two or more tablets or minitablets which may be administered together such that they together provide the necessary amount of all ingredients. “Pharmaceutical composition” and “solid oral dosage form” as used herein include a complete dosage appropriate to a particular administration to a human patient regardless of how it is subdivided so long as it is for substantially simultaneous administration.

In some embodiments, the single dosage form for oral delivery of an API comprises from about 250 mg to about 500 mg of citric acid; from about 11 mg/cm² to about 20 mg/cm² of a water-soluble undercoat; and from about 5 mg/cm² to about 15 mg/cm² of an enteric coating; wherein the API and the citric acid are in the same layer of the dosage form.

In some embodiments, the solid oral dosage form comprises an API intermixed with coated acid particles, the coated acid particles comprising an acid that is coated with a pharmaceutically acceptable protective coating to separate the acid from the API in the solid oral dosage form; an acid resistant protective vehicle; and a water-soluble barrier layer that separates the pH-lowering agent from the acid resistant protective vehicle, wherein the acid resistant protective vehicle is present at a weight from about 7% to about 10%, and wherein the water-soluble barrier layer is present at a weight from about 3.0% to about 12.0%.

In some embodiments, the coated acid particles can be present at a range from about 250 mg to about 500 mg. In some embodiments, the acid include carboxylic acids such as acetylsalicylic, acetic, ascorbic, citric, fumaric, glucuronic, glutaric, glyceric, glycocolic, glyoxylic, isocitric, isovaleric, lactic, maleic, oxaloacetic, oxalosuccinic, propionic, pyruvic, succinic, tartaric, valeric, and the like. In some embodiments, the acid is selected from citric acid, tartaric acid and an acid salt of an amino acid.

In some embodiments, the absorption enhancer comprises an acyl carnitine, or SDS.

In some embodiments, a solid oral dosage form of the present disclosure includes an acid protective vehicle such as an outer layer of enteric coating. Such vehicles are desirable for enhancing bioavailability, but can slow uptake of the API into the bloodstream. In some embodiments, uniform dissolution of the acid protective vehicle in the intestines may be facilitated by keeping the acid of the solid oral dosage form away from the vehicle during its dissolution. This may be accomplished in accordance with the disclosure in one of or more of the following ways. First, the use of a protective water soluble barrier layer between the acid protective vehicle and the acid (pH-lowering agent) of the solid oral dosage form can enhance the more uniform release of all solid oral dosage form in the intestines by permitting most of the acid protective vehicle to dissolve in the intestines before the acid (pH-lowering agent) of the solid oral dosage form is released or otherwise comes in contact with the acid protective vehicle. Otherwise the acid (pH-lowering agent) could adversely affect the dissolution of the acid protective vehicle (which is insoluble in acid environment). This water soluble barrier layer is expected to provide this benefit regardless of the form in which the acid (pH-lowering agent) is supplied, and even when coated acid particles are not present. In some embodiments, the water-soluble barrier layer adds at least 3% to the weight of the solid oral dosage form, exclusive of any acid-protective vehicle. In some embodiments, the water-soluble barrier layer adds from about 3% to about 12% to the weight of the solid oral dosage form, exclusive of any acid-protective vehicle.

Second, the acid (pH-lowering agent) of the composition may be provided in the form of coated acid particles. The coating on these particles is a pharmaceutically acceptable protective coating that is non-acidic and has a solubility in water of at least one gram per 100 milliliters of water at room temperature. In addition to desirably separating the pharmaceutical acid (pH-lowering agent) from the peptide compound, this coating on the acid particles may help protect the solid oral dosage form's acid resistant protective vehicle from the undesirable effects acid can have on quick uniform dissolution of the outer coating in the intestines. This is true even in embodiments of the present disclosure that do not include the protective water soluble barrier layer. In some, but not all, embodiments of the present disclosure, both (1) the protective water soluble barrier layer is present, and (2) the acid (pH-lowering agent) is supplied, at least in part, in the form of coated acid particles.

Likewise, providing acid (pH-lowering agent) to the solid oral dosage form in the form of the foregoing coated acid particles provides numerous advantages that are independent of any effect on enteric coating, and independent of whether or not a protective water soluble barrier layer is used. Such coated acid particles may therefore be used advantageously even in embodiments of the present disclosure that include neither outer coating of acid protective vehicle, nor protective barrier layer. In particular, acid (pH-lowering agent) in the form of coated particles may desirably be thoroughly intermixed with the peptide compound, while undesirable acid-peptide interaction is minimized. Without intending to be bound by theory, this thorough intermixing is believed to facilitate uniform release of each component together so that acid (pH-lowering agent) may better protect the peptide compound, in the intestinal environment, by reducing peptide degradation from the activity of local proteases having neutral or basic pH optima.

In some but not all embodiments, an absorption enhancer is included in a solid oral dosage form to further enhance bioavailability. In some embodiments, coated acid particles, API, absorption enhancer, acid protective vehicle and protective water soluble barrier layer are all present. The use of coated acid particles, in addition to reducing undesirable acid interactions with other components discussed herein, desirably reduces acid interaction with absorption enhancer (when used) or with surfactant (when used).

In some embodiments, coated acid, API, and, optionally, one or more of any optional components discussed herein, e.g. an absorption enhancer, are thoroughly intermixed. The mixture is then coated with both a protective water soluble barrier layer and an outer acid-protective vehicle. In some embodiments, the water soluble barrier layer lies just inside of an acid protective vehicle layer, and separates the vehicle layer from the intermixed remaining contents. In some embodiments, the acid resistant protective vehicle is present at a weight which is from about 3.0% to about 10.0% of the weight of the remainder of the solid oral dosage form. In some embodiments, the acid resistant protective vehicle is present at a weight of about 3.6% of the weight of the remainder of the solid oral dosage form. In some embodiments, the acid resistant protective vehicle is present at a weight of about 7.0% of the weight of the remainder of the solid oral dosage form. In some embodiments, the acid resistant protective vehicle is present at a weight of about 10.0% of the weight of the remainder of the solid oral dosage form. In some embodiments, the water-soluble barrier layer is present at a weight which is from about 6.0% to about 15.0% of the weight of the remainder of the solid oral dosage form. In some embodiments, the water-soluble barrier layer is present at a weight which is from about 7.0% to about 12.0% of the weight of the remainder of the solid oral dosage form. In some embodiments, the water-soluble barrier layer is present at a weight of about 7.0% of the weight of the remainder of the solid oral dosage form. In some embodiments, the water-soluble barrier layer is present at a weight of about 12.0% of the weight of the remainder of the solid oral dosage form.

The acid protective vehicle preferably constitutes an outermost protective layer surrounding the remainder of the solid oral dosage form. The vehicle does not dissolve in the acidic stomach environment, thus protecting the peptide compound from stomach proteases. Without intending to be bound by theory, it is believed that, later, in the basic pH environment of the intestines, the vehicle dissolves quickly without interference from the pharmaceutical acid from which the vehicle is separated by either the barrier layer, or the coating on the acid particles, or both. It is believed that, once the protective vehicle dissolves, the water-soluble barrier layer and the coating surrounding the acid particles release the remaining components of the composition.

The acid (also referred herein as pH lowering agent) is believed to lower the local intestinal pH (where the active agent has been released) to levels below the optimal range for many intestinal proteases. It is believed that this decrease in pH reduces the proteolytic activity of the intestinal proteases, thus affording protection to the peptide compound from potential degradation. The activity of these proteases is diminished by the temporarily acidic environment provided by a solid oral dosage form of the present disclosure. In some embodiments, sufficient acid is provided so that local intestinal pH is lowered temporarily to 5.5 or below. In some embodiments, sufficient acid is provided so that local intestinal pH is lowered temporarily to 4.7 or below. In some embodiments, sufficient acid is provided so that local intestinal pH is lowered temporarily to 3.5 or below. The sodium bicarbonate test, described in the section captioned “the pH-Lowering Agent”, is indicative of the required acid amount. Preferably, conditions of reduced intestinal pH persist for a time period sufficient to protect the API (e.g. peptide, hormone or analogue thereof) from proteolytic degradation until at least some of the peptide compound has had an opportunity to cross the intestinal wall into the bloodstream. Optionally, absorption enhancers, when used, may synergistically promote peptide absorption into the blood while conditions of reduced proteolytic activity prevail. Preferred absorption enhancers and their use are discussed in more detail in a separate section.

Acid and API and, when present, the absorption enhancer, should be released together to the extent possible. The acid is then better able to protect, for example, the peptide compound by reducing degradation of the peptide compound by action of neutral or basic-acting proteases until the peptide compound crosses the intestinal wall into the bloodstream. A concomitant release of absorption enhancer (when used) can further enhance that crossing of the intestinal wall. In a tablet of the present disclosure, additional optional materials, discussed in separate sections herein, aid in forming tablets of appropriate hardness that resist breaking prior to administration, and undergo consistent and complete dissolution at the appropriate time after administration.

According to aspects of the disclosure, the solid dosage form for oral delivery includes from about 10 mg to about 60 mg of a gonadotrophin releasing hormone (GnRH) analogue. According to aspects of the disclosure, the solid dosage form for oral delivery includes from about 40 mg to about 60 mg of a gonadotrophin releasing hormone (GnRH) analogue. According to aspects of the disclosure, the solid dosage form for oral delivery includes from about 45 mg to about 60 mg of a gonadotrophin releasing hormone (GnRH) analogue. According to aspects of the disclosure, the solid dosage form for oral delivery includes from about 50 mg to about 60 mg of a gonadotrophin releasing hormone (GnRH) analogue. In some embodiments, the gonadotrophin releasing hormone (GnRH) analogue is leuprolide or pharmaceutically acceptable salt thereof. In some embodiments, the gonadotrophin releasing hormone (GnRH) analogue is leuprolide acetate. In some embodiments, the solid dosage form is a tablet. In some embodiments, the solid dosage form is a capsule.

According to aspects illustrated herein, there is disclosed a solid oral dosage form that includes a therapeutically effective amount of at least one gonadotrophin releasing hormone (GnRH) analogue; at least one pharmaceutically acceptable pH-lowering agent; an acid resistant protective vehicle; and a water-soluble barrier layer that separates the pH-lowering agent from the acid resistant protective vehicle. In some embodiments, the pH-lowering agent is present in the solid oral dosage form in a quantity which, if the solid oral dosage form were added to ten milliliters of 0.1M aqueous sodium bicarbonate solution, would be sufficient to lower the pH of said solution to no higher than 5.5. In some embodiments, the acid resistant protective vehicle is present at a weight which is from about 3.0% to about 10.0% of the weight of the remainder of the solid oral dosage form. In some embodiments, the acid resistant protective vehicle is present at a weight of about 3.6% of the weight of the remainder of the solid oral dosage form. In some embodiments, the acid resistant protective vehicle is present at a weight of about 7.0% of the weight of the remainder of the solid oral dosage form. In some embodiments, the acid resistant protective vehicle is present at a weight of about 10.0% of the weight of the remainder of the solid oral dosage form. In some embodiments, the water-soluble barrier layer is present at a weight which is from about 7.0% to about 12.0% of the weight of the remainder of the solid oral dosage form. In some embodiments, the water-soluble barrier layer is present at a weight of about 7.0% of the weight of the remainder of the solid oral dosage form. In some embodiments, the water-soluble barrier layer is present at a weight of about 12.0% of the weight of the remainder of the solid oral dosage form. In some embodiments, the solid oral dosage form further includes an absorption enhancer. In some embodiments, the absorption enhancer is a surface active agent. In some embodiments, the surface active agent is absorbable or biodegradable. In some embodiments, the surface active agent is selected from the group consisting of acylcarnitines, phospholipids and bile acids. In some embodiments, the surface active agent is an acyl carnitine. In some embodiments, the solid oral dosage form further comprises a pharmaceutical binder. In some embodiments, the hormone, or analogue thereof, is selected from a gonadotrophin releasing hormone (GnRH) analogue including, but not limited to, triptorelin, leuprorelin and goserelin. In some embodiments, the hormone analogue is leuprorelin. In some embodiments, the acid is selected from citric acid, tartaric acid and an acid salt of an amino acid. In some embodiments, the single oral dosage form is a single tablet or capsule. In some embodiments, an average particle size of the coated acid particles is between 30 mesh and 140 mesh.

In some embodiments, the dosage form comprises a pH lowering agent. In some embodiments, the pH lowering agent comprises citric acid. In some embodiments, the pH lowering agent comprises coated citric acid particles. In some embodiments, the dosage form comprises from about 75 mg to about 500 mg of citric acid. In some embodiments, the dosage form comprises from about 250 mg to about 500 mg of citric acid. In some embodiments, the dosage form comprises from about 75 mg to about 500 mg of citric acid in the form of coated citric acid particles. In some embodiments, the dosage form comprises from about 250 mg to about 500 mg of citric acid in the form of coated citric acid particles. In some embodiments, the dosage form comprises about 250 mg of citric acid in the form of coated citric acid particles.

In some embodiments, the dosage form comprises from about 10 mg/cm² to about 20 mg/cm² of a water-soluble undercoat.

In some embodiments, the dosage form comprises from about 5 mg/cm² to about 15 mg/cm² of an enteric coating.

In some embodiments, the gonadotrophin releasing hormone (GnRH) analogue and the citric acid are in the same layer of the dosage form. In some embodiments, the gonadotrophin releasing hormone (GnRH) analogue and the coated citric acid particles are in the same layer of the dosage form.

In some embodiments, the solid dosage form comprises an absorption enhancer. In some embodiments, the solid dosage form comprises from about 50 mg to about 300 mg absorption enhancer. In some embodiments, the absorption enhancer is an acyl camitine. In some embodiments, the absorption enhancer is lauroyl-L-carnitine. In some embodiments, the gonadotrophin releasing hormone (GnRH) analogue, the citric acid are in the same layer of the dosage form. In some embodiments, the gonadotrophin releasing hormone (GnRH) analogue, the coated citric acid particles are in the same layer of the dosage form.

In some embodiments, the gonadotrophin releasing hormone (GnRH) analogue is selected from the group consisting of triptorelin, leuprorelin and goserelin. In some embodiments, the gonadotrophin releasing hormone analogue is leuprorelin.

In some embodiments, the solid dosage form comprises from about 10 mg to 60 mg of leuprolide (e.g. leuprolide acetate), about 50 mg lauroyl-L-carnitine, about 250 mg citric acid (e.g. coated citric acid particles), wherein the leuprolide, lauroyl-L-carnitine and citric acid are intermixed. In some embodiments, the solid dosage form further comprises one or more of Kollidon® CL (cross-linked polyvinylpyrrolidone, also referred herein as crospovidone), Kollidon® VA64 (vinylpyrrolidone-vinyl acetate copolymer, also referred herein as copovidone), Avicel® PH102 (microcrystalline cellulose), Magnesium stearate, Kollicoat® IR (Ethylene glycol and vinyl alcohol graft copolymer), Eudragit® L30 D55 (methacrylic acid-acrylate copolymer (1:1) dispersion 30%), talc, triethylcitrate.

In some embodiments, the solid dosage is a capsule. In some embodiments, the solid dosage is a tablet.

Non-limiting specific embodiments are described below each of which is considered to be within the present disclosure. Disclosed herein are method of treating a pediatric subject with central precocious puberty (CPP). Leuprolide or a pharmaceutically acceptable salt thereof for use in a method of treating a pediatric subject with central precocious puberty (CPP) is disclosed herein.

Embodiment 1. A method of treating a pediatric subject with central precocious puberty (CPP), the method comprising:
administering orally to the pediatric subject having CPP a total daily dose of from about 20 mg to about 60 mg leuprolide or a pharmaceutically acceptable salt thereof in a solid dosage form, wherein the pediatric subject has a body weight of less than 25 kg.

Embodiment 2. A method of treating a pediatric subject with central precocious puberty (CPP), the method comprising:
administering orally to the pediatric subject having CPP a total daily dose of from about 20 mg to about 40 mg leuprolide or a pharmaceutically acceptable salt thereof in a solid dosage form, wherein the pediatric subject has a body weight of less than 25 kg.

Embodiment 3. A method of treating a pediatric subject with central precocious puberty (CPP), the method comprising:
administering orally to the pediatric subject having CPP a total daily dose of from about 40 mg to about 60 mg leuprolide or a pharmaceutically acceptable salt thereof in a solid dosage form, wherein the pediatric subject has a body weight of less than 25 kg.

Embodiment 4. A method of treating a pediatric subject with central precocious puberty (CPP), the method comprising:
administering orally to the pediatric subject having CPP a total daily dose of from about 40 mg to about 80 mg leuprolide or a pharmaceutically acceptable salt thereof in a solid dosage form, wherein the pediatric subject has a body weight ranging from 25 kg and 37.5 kg.

Embodiment 5. A method of treating a pediatric subject with central precocious puberty (CPP), the method comprising:
administering orally to the pediatric subject having CPP a total daily dose of from about 40 mg to about 60 mg leuprolide or a pharmaceutically acceptable salt thereof in a solid dosage form, wherein the pediatric subject has a body weight ranging from 25 kg and 37.5 kg.

Embodiment 6. A method of treating a pediatric subject with central precocious puberty (CPP), the method comprising:
administering orally to the pediatric subject having CPP a total daily dose of from about 60 mg to about 80 mg leuprolide or a pharmaceutically acceptable salt thereof in a solid dosage form, wherein the pediatric subject has a body weight ranging from 25 kg and 37.5 kg.

Embodiment 7. A method of treating a pediatric subject with central precocious puberty (CPP), the method comprising:
administering orally to the pediatric subject having CPP a total daily dose from about 60 mg to about 120 mg leuprolide or a pharmaceutically acceptable salt thereof in a solid dosage form, wherein the pediatric subject has a body weight of greater than 37.5 kg.

Embodiment 8. A method of treating a pediatric subject with central precocious puberty (CPP), the method comprising:
administering orally to the pediatric subject having CPP a total daily dose from about 60 mg to about 90 mg leuprolide or a pharmaceutically acceptable salt thereof in a solid dosage form, wherein the pediatric subject has a body weight of greater than 37.5 kg.

Embodiment 9. A method of treating a pediatric subject with central precocious puberty (CPP), the method comprising:
administering orally to the pediatric subject having CPP a total daily dose from about 90 mg to about 120 mg leuprolide or a pharmaceutically acceptable salt thereof in a solid dosage form, wherein the pediatric subject has a body weight of greater than 37.5 kg.

Embodiment 10. The method of any one of embodiments 1 to 9, wherein the administration reduces mean ratio of bone age to chronological age of the subject at the time of measurement to about 1.4 or lower over twelve month treatment period.

Embodiment 11. The method of any one of embodiments 1 to 9, wherein the administration reduces mean ratio of bone age to chronological age of the subject at the time of measurement to about 1 over about a sixty month treatment period.

Embodiment 12. A method of treating a pediatric subject with central precocious puberty (CPP), the method comprising:
administering orally to the pediatric patient having CPP a solid dosage from comprising a therapeutically effective amount of leuprolide or a pharmaceutically acceptable salt thereof,
wherein the therapeutically effective amount of leuprolide or a pharmaceutically acceptable salt thereof is from about 20 mg to about 120 mg, and
wherein administration reduces mean ratio of bone age to chronological age of the subject at the time of measurement to about 1.4 or lower over twelve month treatment period.

Embodiment 13. A method of treating a pediatric patient with central precocious puberty (CPP), the method comprising:
administering orally to the pediatric patient having CPP a solid dosage form comprising between about 10 mg to about 60 mg of leuprolide or a pharmaceutically acceptable salt thereof,
wherein administration of the solid dosage form for a suitable period of time reduces the mean bone age to chronological age of the subject at the time of measurement to about 1.4 or lower over about a twelve-month treatment period.

Embodiment 14. The method of embodiment 12 or 13, wherein the solid dosage form comprises from about 10 mg to about 60 mg leuprolide or a pharmaceutically acceptable salt thereof.

Embodiment 15. The method of embodiment 12 or 13, wherein the solid dosage form comprises from about 20 mg to about 60 mg leuprolide or a pharmaceutically acceptable salt thereof.

Embodiment 16. The method of embodiment 12 or 13, wherein the solid dosage form comprises from about 40 mg to about 60 mg leuprolide or a pharmaceutically acceptable salt thereof.

Embodiment 17. The method of embodiment 12 or 13, wherein the solid dosage form comprises from about 50 mg to about 60 mg leuprolide or a pharmaceutically acceptable salt thereof.

Embodiment 18. The method of any preceding embodiment wherein the solid dosage form comprises about 60 mg of leuprolide or a pharmaceutically acceptable salt thereof.

Embodiment 19. The method of any preceding embodiment wherein the solid dosage form comprises about 50 mg of leuprolide or a pharmaceutically acceptable salt thereof.

Embodiment 20. The method of any preceding embodiment wherein the solid dosage form comprises about 45 mg of leuprolide or a pharmaceutically acceptable salt thereof.

Embodiment 21. The method of any preceding embodiment wherein the solid dosage form comprises more than about 40 mg and less than about 60 mg of leuprolide acetate.

Embodiment 22. The method of any preceding embodiment, wherein the total daily dose is administered once daily.

Embodiment 23. The method of embodiment 22, wherein the once daily dose is administered in the morning before consumption of food.

Embodiment 24. The method of any preceding embodiment, wherein half of the total daily dose is administered twice daily.

Embodiment 25. The method embodiment 24, wherein the two daily doses are administered at about 10 hours to about 14 hours interval.

Embodiment 26. The method of embodiment 24, wherein the first dose of the two doses is administered in the morning before consumption of food.

Embodiment 27. The method embodiment 24, wherein the second dose of the two doses is administered up to 6 hours after consumption of food.

Embodiment 28. The method of any preceding embodiment wherein the solid dosage form is administered once daily.

Embodiment 29. The method of any preceding embodiment wherein the solid dosage form is administered twice daily.

Embodiment 30. The method of any preceding embodiment, comprising administering the solid dosage form for a time period of about 6 months, of about 12 months, of about 18 months, of about 24 months, or longer.

Embodiment 31. The method of any preceding embodiment wherein the solid dosage form is a tablet or a capsule.

Embodiment 32. The method of any preceding embodiment wherein the solid dosage form is a tablet comprising (a) leuprolide or a pharmaceutically acceptable salt thereof; (b) an absorption enhancer; (c) coated acid particles intermixed with the leuprolide or a pharmaceutically acceptable salt thereof, wherein the coating separates the acid from the leuprolide or a pharmaceutically acceptable salt thereof in the tablet; (d) an outer layer of an acid-resistant enteric coating effective to transport the tablet through the stomach of the patient while preventing contact between the leuprolide or a pharmaceutically acceptable salt thereof and stomach proteases; and (e) a water soluble barrier layer beneath the outer layer of enteric coating that separates the enteric coating from the coated acid particles.

Embodiment 33. The method of any preceding embodiment wherein the coated acid particles comprise coated citric acid particles.

Embodiment 34. The method of any preceding embodiment wherein the pharmaceutically acceptable salt of leuprolide is leuprolide acetate.

Embodiment 35. The method of any preceding embodiment wherein the absorption enhancer comprises an acyl carnitine.

Embodiment 36. The method of any preceding embodiment wherein the absorption enhancer comprises lauroyl carnitine.

Embodiment 37. The method of any preceding embodiment wherein the administration reduces mean ratio of bone age to chronological age of the subject at the time of measurement to about 1 over about a sixty month treatment period.

Embodiment 38. Leuprolide or a pharmaceutically acceptable salt thereof for use in a method of treating a pediatric subject with central precocious puberty (CPP), the method comprising:
administering orally to the pediatric subject having CPP a total daily dose of from about 20 mg to about 60 mg of the leuprolide or pharmaceutically acceptable salt thereof in a solid dosage form, wherein the pediatric subject has a body weight of less than 25 kg.

Embodiment 39. Leuprolide or a pharmaceutically acceptable salt thereof for use in a method of treating a pediatric subject with central precocious puberty (CPP), the method comprising:
administering orally to the pediatric subject having CPP a total daily dose of from about 20 mg to about 40 mg of the leuprolide or pharmaceutically acceptable salt thereof in a solid dosage form, wherein the pediatric subject has a body weight of less than 25 kg.

Embodiment 40. Leuprolide or a pharmaceutically acceptable salt thereof for use in a method of treating a pediatric subject with central precocious puberty (CPP), the method comprising:
administering orally to a pediatric subject having CPP a total daily dose of from about 40 mg to about 60 mg of the leuprolide or pharmaceutically acceptable salt thereof in a solid dosage form, wherein the pediatric subject has a body weight of less than 25 kg.

Embodiment 41. Leuprolide or a pharmaceutically acceptable salt thereof for use in a method of treating a pediatric subject with central precocious puberty (CPP), the method comprising:
administering orally to the pediatric subject having CPP a total daily dose of from about 40 mg to about 80 mg the leuprolide or pharmaceutically acceptable salt thereof in a solid dosage form, wherein the pediatric subject has a body weight ranging from 25 kg and 37.5 kg.

Embodiment 42. Leuprolide or a pharmaceutically acceptable salt thereof for use in a method of treating a pediatric subject with central precocious puberty (CPP), the method comprising:
administering orally to the pediatric subject having CPP a total daily dose of from about 40 mg to about 60 mg of the leuprolide or pharmaceutically acceptable salt thereof in a solid dosage form, wherein the pediatric subject has a body weight ranging from 25 kg and 37.5 kg.

Embodiment 43. Leuprolide or a pharmaceutically acceptable salt thereof for use in a method of treating a pediatric subject with central precocious puberty (CPP), the method comprising:
administering orally to the pediatric subject having CPP a total daily dose of from about 60 mg to about 80 mg of the leuprolide or pharmaceutically acceptable salt thereof in a solid dosage form, wherein the pediatric subject has a body weight ranging from 25 kg and 37.5 kg.

Embodiment 44. Leuprolide or a pharmaceutically acceptable salt thereof for use in a method of treating a pediatric subject with central precocious puberty (CPP), the method comprising:
administering orally to the pediatric subject having CPP a total daily dose from about 60 mg to about 120 mg of the leuprolide or pharmaceutically acceptable salt thereof in a solid dosage form, wherein the pediatric subject has a body weight of greater than 37.5 kg.

Embodiment 45. Leuprolide or a pharmaceutically acceptable salt thereof for use in a method of treating a pediatric subject with central precocious puberty (CPP), the method comprising:
administering orally to the pediatric subject having CPP a total daily dose from about 60 mg to about 90 mg of the leuprolide or a pharmaceutically acceptable salt thereof in a solid dosage form, wherein the pediatric subject has a body weight of greater than 37.5 kg.

Embodiment 46. Leuprolide or a pharmaceutically acceptable salt thereof for use in a method of treating a pediatric subject with central precocious puberty (CPP), the method comprising:
administering orally to the pediatric subject having CPP a total daily dose from about 90 mg to about 120 mg of the leuprolide or pharmaceutically acceptable salt thereof in a solid dosage form, wherein the pediatric subject has a body weight of greater than 37.5 kg.

Embodiment 47. Leuprolide or a pharmaceutically acceptable salt thereof for use in a method of treating a pediatric subject with central precocious puberty (CPP), the method comprising:
administering orally to the pediatric subject having CPP a solid dosage from comprising a therapeutically effective amount of leuprolide or a pharmaceutically acceptable salt thereof,
wherein the therapeutically effective amount of the leuprolide or pharmaceutically acceptable salt thereof is from about 20 mg to about 120 mg, and
wherein administration reduces mean ratio of bone age to chronological age of the pediatric subject at the time of measurement to about 1.4 or lower over twelve month treatment period.

Embodiment 48. Leuprolide or a pharmaceutically acceptable salt thereof for use in a method of treating a pediatric subject with central precocious puberty (CPP), the method comprising:
administering orally to the pediatric subject having CPP a solid dosage form comprising between about 10 mg to about 60 mg of the leuprolide or pharmaceutically acceptable salt thereof,
wherein administration of the solid dosage form for a suitable period of time reduces the mean bone age to chronological age of the pediatric subject at the time of measurement to about 1.4 or lower over about a twelve-month treatment period.

As various changes can be made in the above-described subject matter without departing from the scope and spirit of the present disclosure, it is intended that all subject matter contained in the above description, be interpreted as descriptive and illustrative of the present disclosure. Many modifications and variations of the present disclosure are possible in light of the above teachings. Accordingly, the present description is intended to embrace all such alternatives, modifications and variances which fall within the scope of the appended claims.

All documents cited or referenced herein and all documents cited or referenced in the herein cited documents, together with any manufacturer's instructions, descriptions, product specifications, and product sheets for any products mentioned herein or in any document incorporated by reference herein, are hereby incorporated by reference, and may be employed in the practice of the disclosure.

### EXAMPLES

The present disclosure is further illustrated by reference to the following Examples. However, it should be noted that these Examples, like the embodiments described above, are illustrative and are not to be construed as restricting the scope of the invention in any way.

### Example 1: Study to Evaluate the Absorption and Pharmacokinetic (PK) Profiles of Two Doses and Two Dosing Regimens of Leuprolide Acetate Oral Tablet Under Fasting and Fed Conditions in Healthy Female Volunteers

AN OPEN-LABEL, FIVE-PERIOD, CROSSOVER, SINGLE-
SEQUENCE STUDY TO EVALUATE THE ABSORPTION
AND PHARMACOKINETIC (PK) PROFILES OF TWO
DOSES AND TWO DOSING REGIMENS OF LEUPROLIDE
ACETATE ORAL TABLET UNDER FASTING AND FED
CONDITIONS IN HEALTHY FEMALE VOLUNTEERS.

| | |
|---|---|
| Protocol No.: | LOPDT-PH1-02 |
| Study Drugs: | Treatment A: Leuprolide Acetate Oral Tablet, |
| | 40 mg, BID (fasted) |
| | Treatment B: Leuprolide Acetate Oral Tablet, |
| | 60 mg, BID (fasted); |
| | Treatment C: Leuprolide Acetate Oral Tablet, |
| | 120 mg, QD (fasted); |
| | Treatment D: Leuprolide Acetate Oral Tablet, |
| | 60 mg, BID (fed, 2-hour interval) |
| | Treatment E: Leuprolide Acetate Oral Tablet, |
| | 60 mg, BID (fed, 1-hour interval) |
| Study Phase: | Phase 1 |

### Objectives:

This trial was intended to be a bridging pharmacokinetic (PK) study to historical PK data for another currently marketed formulation of leuprolide acetate—Lupron Depot® 3.75 mg.

The most important objective of this study was to provide adequate PK evaluations across the selected single doses and daily dosing regimens to support development of the Leuprolide Oral Tablet with projected overall drug exposure similar to that reported for the 3.75 mg of Lupron Depot.

Another objective was to describe the dose-proportionality of Leuprolide Oral Tablet within the 80 mg-120 mg targeted daily dosing and within the 40 mg-120 mg targeted single dose.

Finally, the food effect pharmacokinetics of the Leuprolide Oral Tablet were also to be assessed.

Fasting conditions in this study correspond to morning doses taken after an overnight (at least 10 hour) fast. No food consumption was allowed until 4 hours after dosing. The same conditions applied if the regimen was a once-daily dosing schedule. Evening doses in this condition were administered at least 4 hours after food consumption.

Under the fed conditions the morning dose was taken after an overnight fast, but food consumption was either 2 hours or 1 hour after the morning medication dose. The evening dose, when applicable, was administered either 2 hours or 1 hour after food consumption.

### Primary Study Objectives:

1) To provide comparative evaluation of the PK profile of leuprolide acetate in healthy female volunteers following administration of 80-mg and 120 mg total daily doses of Leuprolide Oral Tablets under fasting conditions.
2) To provide comparative evaluation of the PK profile of leuprolide acetate in healthy female volunteers following administration of a total daily dose of 120-mg of Leuprolide Oral Tablets administered as QD and BID regimens under fasting conditions.
3) To assess the dose-proportionality of a single dose of the Leuprolide Oral Tablet within the 40 mg-120 mg range under fasting conditions.
4) To assess the food effect on the PK profile of a single 60-mg dose administered in the morning under both fasting and fed conditions (2 hours before breakfast and 1 hour before breakfast)
5) To assess the food effect on the PK profile of a 60-mg BID dosing administered under both fasting and fed conditions (2 hours before breakfast/2 hours after dinner or 1 hour before breakfast/1 hour after dinner).

### Secondary Study Objective:

To evaluate safety and tolerability of Leuprolide Oral Tablets leuprolide acetate administered within the 80 mg-120 mg daily dosing range under fasting and fed conditions in healthy female volunteers.

### Methodology:

This was a single-center, open-label, five-period, crossover, single-sequence, dose-proportionality and food-effect study to evaluate the PK and safety profiles of two doses and two dosing regimens of Leuprolide Oral Tablet under fasting and fed conditions in healthy female volunteers.

This study evaluated Leuprolide Oral Tablets (Ovarest®) utilizing Enteris' Oral Peptide Delivery Technology.

The following dosing regimens were tested in this study:
Treatment “A”: Leuprolide Oral Tablet (Ovarest®), 40 mg, administered twice daily (BID), 12 hours apart under fasting conditions (also referred herein as food-intake restrictions, the dosing is scheduled ≈4 hours before breakfast and ≈4 hours after dinner)
Treatment “B”: Leuprolide Oral Tablet (Ovarest®), 60 mg, administered twice daily (BID, under fasting conditions (the dosing is scheduled ≈4 hours before breakfast and ≈4 hours after dinner)
Treatment “C”: Leuprolide Oral Tablet (Ovarest®), 120 mg, administered once daily (QD), 2×60 mg tablets in the morning under fasting conditions (the dosing is scheduled ≈4 hours before breakfast)
Treatment “D”: Leuprolide Oral Tablet (Ovarest®), 60 mg, administered twice daily (BID), 12 hours apart under fed conditions (the dosing is scheduled ≈2 hours before breakfast and ≈2 hours after dinner)
Treatment “E”: Leuprolide Oral Tablet (Ovarest®), 60 mg, administered twice daily (BID), 12 hours apart under fed conditions (the dosing is scheduled ≈1 hour before breakfast and ≈1 hour after dinner)

Treatments “A”, “B”, “C”, “D”, and “E” were administered in sequential order. Since ABCDE was the only treatment sequence, the study was non-randomized.

The study has consisted of the following 5 periods:
Period 1 (Study Days 1-2): Treatment “A”
Period 2 (Study Days 3-4): Treatment “B”
Period 3 (Study Days 5-6): Treatment “C”
Period 4 (Study Days 7-8): Treatment “D”
Period 5 (Study Days 9-10): Treatment “E”

Study Days 1, 3, 5, 7, and 9 were dosing days, and Study Days 2, 4, 6, 8, and 10 are post-dosing days, Treatment (dosing) periods were separated by a 1-day washout. (a respective post-dosing day).

Subjects were confined to the clinical study unit from at least 11 hours prior to the drug administration until the 36-hour post-dose blood draw in the fifth period.

A number of PK parameters (Cmax, Tmax, AUCs and trough levels) were calculated for each subject for each treatment period. Key assessments of the PK profile were based on the following metrics:
(1) The total daily exposure estimated by the AUC₀₋₂₄
(2) The exposure over the 12 post-dosing hours estimated by the AUC₀₋₁₂ after the first dosing (BID regimen) or a single dosing (QD regimen).
(3) “Trough” concentration level at a 24-hour timepoint (C24) for the QD regimen and at a 36-hour timepoint (C36) for the BID regimens.

### Number of Subjects (Planned and Analyzed):

A total of 22 healthy premenopausal female volunteers were planned to be enrolled in this study. Approximately 20 subjects were expected to complete all five dosing periods.

Twenty-two (22) subjects were enrolled in the study. All of them were dosed and considered evaluable for safety analyses. One (1) subject was considered as early withdrawal for a reason unrelated to safety. Twenty-one (21) subjects completed the study and were included in the primary PK population.

### Diagnosis and Main Criteria for Inclusion:

Subjects had to be healthy, premenopausal females, aged ≥18 and ≤49 years, with body mass index (BMI) ≥18.0 and <32.0 kg/m² and willing to use a non-hormonal method of contraception during the study if of childbearing potential. All subjects had to be in compliance with the inclusion and exclusion criteria described in the protocol and were judged eligible for enrolment in this study based on medical and medication histories, demographic data (including sex, age, race, ethnicity, body weight [kg], height [cm], and BMI [kg/m²]), vital signs measurements, a 12-lead electrocardiogram (ECG), a physical examination, a urine drug screen, an alcohol breath test, a pregnancy test, and clinical laboratory tests (biochemistry, hematology, serology, urinalysis, human immunodeficiency virus [HIV], hepatitis C [HCV] antibodies, and hepatitis B surface antigen [HBsAg]).

Treatment Code

| | | | |
|---|---|---|---|
| Parameter | Treatment A | Treatment B, | Treatment C |
| | (administered BID, | Treatment D, | (administered QD) |
| | 12 hours apart) | Treatment E | |
| | | (administered BID | |
| | | 12 hours apart) | |
| Product | Ovarest ® | Ovarest ® | Ovarest ® |
| Strength | 40 mg | 60 mg | 60 mg |
| Dosage form | Tablet | Tablet | Tablet |
| Dose | 1 × 40 mg | 1 × 60 mg | 2 × 60 mg |
| administered | | | |
| Route of | Oral | Oral | Oral |
| administration | | | |
| Inactive | Citric acid DC F20 | Citric acid DC F20 | Citric acid DC F20 |
| ingredients | (250 mg), Lauroyl-L- | (250 mg), Lauroyl-L- | (250 mg), Lauroyl-L- |
| | carnitine (50 mg), | carnitine (50 mg), | carnitine (50 mg), |
| | Crospovidone (19 mg), | Crospovidone (22 mg), | Crospovidone (22 mg), |
| | Copovidone (38 mg), | Copovidone (44 mg), | Copovidone (44 mg), |
| | Microcrystalline | Microcrystalline | Microcrystalline |
| | cellulose (79 mg), | cellulose (121 mg), | cellulose (121 mg), |
| | Magnesium stearate | Magnesium stearate | Magnesium stearate |
| | (2 mg), Ethylene | (3 mg), Ethylene | (3 mg), Ethylene |
| | glycol and vinyl | glycol and vinyl | glycol and vinyl |
| | alcohol graft | alcohol graft | alcohol graft |
| | copolymer (58 mg), | copolymer (66 mg), | copolymer (66 mg), |
| | Methacrylic acid | Methacrylic acid | Methacrylic acid |
| | copolymer dispersion | copolymer dispersion | copolymer dispersion |
| | and Methacrylic acid - | and Methacrylic acid - | and Methacrylic acid - |
| | ethyl acrylate | ethyl acrylate | ethyl acrylate |
| | copolymer, (1:1) 30% | copolymer, (1:1) 30% | copolymer, (1:1) 30% |
| | Dispersion (36 mg), | Dispersion (41 mg), | Dispersion (41 mg), |
| | Talc (9 mg), | Talc (10 mg), | Talc (10 mg), |
| | Triethylcitrate | Triethylcitrate | Triethylcitrate |
| | (4 mg), and | (4 mg), and | (4 mg), and |
| | Purified water. | Purified water. | Purified water. |
| Manufacturer | Enteris BioPharma Inc | Enteris BioPharma Inc | Enteris BioPharma Inc |
| Lot/Batch No. | PT8024A-040-2101 | PT8024A-060-2101 | PT8024A-060-2101 |

### Duration of Treatment:

The total study duration, from screening visit to study exit evaluations was 1 month or less.

### Pharmacokinetic (PK) Blood Draws:

### Criteria for Evaluation:

### Pharmacokinetics:

At each BID treatment period (Periods 1, 2, 4, and 5), twenty-two (22) serial serum samples for the pharmacokinetic evaluations were drawn at baseline (immediately prior to the first oral dosing), 0 time) and at 0.5, 1, 1.5, 2, 2.5, 3, 3.5, 4, 4.5, 5, 6, 7, 8, 10, 12 (immediately prior to the second dosing), 16, 20, 24, 28, 32, and 36 hours following the first oral dosing.

At the QD treatment period (Period 3) nineteen (19) serial serum samples for the pharmacokinetic evaluations were drawn at baseline (immediately prior to a single oral dosing), 0 time) and at 0.5, 1, 1.5, 2, 2.5, 3, 3.5, 4, 4.5, 5, 6, 7, 8, 10, 12, 16, 20, and 24 hours following the single oral dosing.

The blood samples were appropriately stored until assayed for leuprolide acetate concentration levels. Serum concentrations of leuprolide acetate were determined via a validated bioanalytical method. Sensitivity and specificity of the employed bioanalytical method are described in the study report.

The following PK parameters were evaluated for each subject for each treatment period:
Maximum concentration level (Cmax) for 12-hour and 24-hour periods;
Time to maximum concentration level (Tmax) for 12-hour and 24-hour periods;
“Trough” concentration level at a 24-hour timepoint (C24) for the QD regimen and at a 36-hour timepoint (C36) for the BID regimens.
Area under the concentration versus time curve from 0 hours to 12 hours (AUC0-12);
Area under the concentration versus time curve from 0 hours to 24 hours (AUC0-24);
Area under the concentration versus time curve from 0 hours to 36 hours (AUC0-36)—for the BID regimens only.
Area under the concentration versus time curve from 0 hours to last available observation (AUC0-t);
Area under the concentration versus time curve from 0 hours to infinity (AUC0-inf);
Elimination half-life (t½)
Elimination rate constant, (Kel).

### Safety:

Safety evaluations were based on changes from screening to last assessment (study exit) in physical examinations, changes in clinical laboratory test results, treatment-induced changes in vital signs during each treatment period, and by the incidence and severity of treatment-emergent adverse events (TEAEs) reported. TEAEs were assigned to the study period in which they occurred: pre-dose, Period 1, Period 2, Period 3, Period 4, and Period 5.

### Statistical Methods:

### Pharmacokinetic Analyses:

The primary statistical analyses of PK data were conducted on the population of subjects completing five treatment periods. Supporting evaluations were performed for the cohort of subjects completing at least one treatment period. Since only one subject discontinued the study early (completed four out of five treatment periods), these populations were nearly identical. Therefore, the PK analyses were performed for the latter dataset only which was designated as a Primary PK population.

Pharmacokinetic metrics were summarized by treatment received using means, geometric means, standard deviations, coefficients of variation, medians, and minimum and maximum values.

In the comparative statistical evaluation of the dose-proportionality of the single dose of Leuprolide Oral Tablets, the fasted morning 40 mg oral dose (Treatment “A”) was considered as a reference formulation while the fasted morning 60 mg oral dose, (Treatment “B”) and the fasted morning 120 mg oral dose, (Treatment “C”) were considered as test formulations. Note: the pharmacokinetic parameters for Treatment “B” and Treatment “C” were dose-normalized (to the 40 mg oral tablet).

In the comparative statistical evaluation of the dose-proportionality of the total daily dose of Leuprolide Oral Tablets, the fasted 40 mg BID oral dose (Treatment “A”) was considered as a reference formulation while the 60 mg BID oral dose (Treatment “B”) and the fasted 120 mg QD oral dose, (Treatment “C”) were considered as test formulations. Note: the pharmacokinetic parameters for Treatment “B” and Treatment “C” were dose-normalized (to the 80 mg total daily dose).

In the comparative statistical evaluation of the dosing regimens (BID vs. QD) of Leuprolide Acetate Oral Tablets, the fasted 120 mg oral dose, QD (Treatment “C”) was considered as a reference formulation while the fasted 60 mg oral dose, BID (Treatment “B”) was considered as a test formulation.

In the comparative statistical evaluation of the food effect, the fasted 60 mg oral dose, BID (Treatment “B”) was considered as a reference formulation while the fed regimens of 60 mg oral dose, BID (Treatment “D” and Treatment “E”) were considered as test formulations.

Noted evaluations were performed for the total daily exposure as well as a 12-hour exposure following the first (BID) or a single (QD) dose of the study drug.

Comparative evaluations were performed across all other PK parameters, including Cmax, AUCs and “trough” concentrations.

All statistical comparisons were performed employing treatment ratios of respective PK metrics estimated for individual subjects.

Leuprolide concentrations at each time point were summarized descriptively and graphed by treatment versus time for individual subjects and for the mean and, if considered appropriate, for geometric mean and median values.

### Safety Endpoints:

### Safety Analyses:

Study discontinuation information, changes in the physical findings, and treatment-emergent changes in vital signs, and clinical laboratory test results were summarized.

The subject incidence of treatment-emergent adverse events (TEAEs) was reported by the treatment received. The Medical Dictionary for Regulatory Activities (MedDRA®) dictionary was used to classify all TEAEs reported during the study by System Organ Class (SOC) and Preferred Term (PT). Incidence of subjects who experienced TEAEs was presented by treatment group, by SOC, PT, by severity and by Investigator-assessed causality (relationship to study drug).

Summaries of vitals signs were presented for each evaluation time point, Treatment induced changes in vital signs were summarized

TABLE 1
Summary of Major Pharmacokinetic Parameters (Primary PK Population)

| | Arithmetic Mean | | Median | Arithmetic Mean | | Median |
|---|---|---|---|---|---|---|
| | (SD) | | (Min-Max) | (SD) | | (Min-Max) |
| | AUC₀₋₂₄ | Cmax₀₋₂₄ | Tmax₀₋₂₄ | AUC₀₋₁₂ | Cmax₀₋₁₂ | Tmax₀₋₁₂ |
| | (h*ng/mL) | (ng/mL) | (hour) | (h*ng/mL) | (ng/mL) | (hour) |
| 40 mg BID (fasted) | 255.4 | 93.4 | 4.00 | 169.9 | 89.6 | 3.25 |
| Treatment A (N = 22) | (192.3) | (96.5) | (2-24) | (177.0) | (98.9) | (2-12) |
| 60 mg BID (fasted) | 372.9 | 114.8 | 3.00 | 213.7 | 102.7 | 2.50 |
| Treatment B (N = 22) | (265.0) | (87.9) | (0-24) | (167.8) | (80.7) | (0-7) |
| 120 mg QD (fasted) | 534.7 | 230.0 | 3.00 | 497.4 | 227.7 | 3.00 |
| Treatment C (N = 22) | (395.2) | (166.6) | (0-24) | (378.3) | (169.5) | (0-7) |
| 60 mg BID (fed, 2 h) | 224.4 | 81.1 | 3.50 | 149.6 | 74.6 | 3.00 |
| Treatment D (N = 22) | (214.4) | (92.3) | (0-24) | (185.7) | (93.6) | (0-12) |
| 60 mg BID (fed, 1 h) | 185.6 | 60.7 | 4.50 | 87.4 | 43.6 | 2.00 |
| Treatment E (N = 21) | (170.9) | (65.0) | (0-24) | (130.9) | (69.3) | (0-4.5) |

The PK study results confirm adequate delivery of leuprolide acetate using the leuprolide oral solid dosage forms disclosed herein.

After administration of the 60 mg BID (fasted) regimen (Treatment B), the total daily exposure (as measured by mean AUC₀₋₂₄) was 1.4 times greater when compared to the 40 mg BID (fasted) regimen (Treatment A) which is close to 1.5 times difference in the administered dose. This results suggest the dose proportionality of leuprolide oral tablets.

The data suggests significant food effect on the PK metrics. After administration of the 60 mg BID (fasted) regimen (Treatment B), the total daily exposure was 1.7 times and 2.0 times greater when compared to the same dose administered under fed conditions (Treatment D and Treatment E, respectively). The same phenomenon is observed for a single (first) dose: 12-hour exposure was 1.4 times and 2.4 times greater for Treatment B vs. Treatment D and Treatment E, respectively. Approximately the same treatment differences for the noted comparisons were observed for Cmax reported after the first dose (Cmax₀₋₁₂).

Adequate drug delivery was confirmed by concentration levels at the end of the 24-hour evaluation interval. The incidence of drug concentrations exceeding below the limit of quantification (BLQ) at that time point was 100%; 95.5%, 95.5%; 86.4%, and 85.7% for Treatment A, Treatment B, Treatment C, Treatment D, and Treatment E, respectively.

Notably, the leuprolide oral solid dosage forms disclosed herein delivered more drug than reported for other studied or marketed leuprolide formulations (intravenous, subcutaneous and intramuscular). Both overall drug exposure (estimated by AUC) and maximum plasma concentration (Cmax) has approached or exceeded other studied or marketed leuprolide formulations.

### Safety:

Overall, there were no notable safety concerns related to the number and subject incidence of the Treatment-Emergent Adverse Events (TEAEs) as well as treatment-induced changes in clinical laboratory test results and vital signs.

There were no Serious Adverse Events (SAEs), no severe TEAEs, and no safety or laboratory-related TEAEs reported in this study. There were no discontinuations due to TEAEs.

A total of 76 adverse events were considered to be TEAEs, which were reported by 17 (77.3%) of the 22 subjects who received at least one dose of the study medication. The subject incidence of TEAEs across the treatment groups was as follows: 27.3% (6 out of 22 subjects) in Treatment A, 31.8% (7 out of 22 subjects) in Treatment B, 40.9% (9 out of 22 subjects) in Treatment C, 40.9% (9 out of 22 subjects) in Treatment D, and 42.9% (9 out of 21 subjects) in Treatment E.

Across all Treatments, the most common TEAEs was headache (a total of 15 events, with the incidence ranging from 14.3% in Treatment B to 26.7% in Treatment E), dizziness (a total of 8 events, with the incidence ranging from 6.7% in Treatment E to 13.3% in Treatment C), and nausea (a total of 7 events, with the incidence ranging from 0% in Treatment B to 13.3% in Treatment C and Treatment E),

Sixty-five out of 76 TEAEs (85.5%) were mild in severity with the respective percentage ranging from 78.6% in Treatment B to 93.7% in Treatment A. Remaining TEAEs were moderate in severity.

Twenty-two out of 76 TEAEs (29.0%) were unrelated to study drug with the respective percentage ranging from 12.5% in Treatment A to 50.0% in Treatment B.

No notable safety issues were observed for clinical laboratory tests results and treatment-induced changes in vital signs. The study drugs were well tolerated by the subjects.

### Overall Conclusion:

Administration of a 80 mg-120 mg total daily dose of Leuprolide Acetate Oral Tablets to healthy females was safe and well tolerated. Estimates of the PK metrics in conjunction with historical data for marketed formulations of leuprolide acetate will enable selection of potentially safe and efficacious oral doses for testing in future clinical studies.

### Example 2: Administration in Children

The approved doses of leuprolide (SC or IM injectables) for the different indications are shown in Table 2 below:

TABLE 2

| Indication | Population | Approved Doses of Leuprolide |
|---|---|---|
| Endometriosis/ | Adult Women | 1 mg daily SC |
| uterine fibroids | ″ | 3.75 mg monthly IM |
| | ″ | 11.25 mg every 3 months IM |
| Advanced prostate | Adult Men | 1 mg daily SC |
| cancer | ″ | 7.5 mg monthly IM |
| | ″ | 22.5 mg every 3 months IM |
| | ″ | 30 mg every 4 months IM |
| | ″ | 45 mg every 6 months IM |
| Central precocious | Children | 0.05 mg/kg body weight daily SC |
| puberty | | (titrate dose upward based on body |
| | | weight and age) |
| | ″ | 7.5/11.25/15 mg monthly IM |
| | ″ | 11.25/30 mg every 3 months IM |
| | | (titrate dose upward based on body |
| | | weight) |
| | ″ | 45 mg every 6 months SC |

As shown above, the dose requirements that have been shown as necessary to achieve and maintain suppression of children with central precocious puberty into the pre-pubertal range are higher than those required in adult women to treat benign gynecologic conditions, and even greater in some cases than are required in adult men with advanced prostatic carcinoma. Without being bound to the theory, this may be due, in part, to more active metabolism of GnRH agonists in the pediatric population.

It is generally accepted that many women suffering from pelvic pain due to endometriosis, and some with excessive menstrual bleeding due to uterine fibroids, can achieve adequate relief and control of their symptoms with doses of leuprolide which are sufficient to suppress their estradiol levels into the range representative of the early follicular phase of the menstrual cycle, typically ˜40 pg/mL. Yet other patients may require more profound degrees of suppression, into the menopausal range, ˜20 pg/mL.

Based on this observation, the ability to achieve complete suppression of estradiol to <20 pg/mL with one or more of the dose regimens described herein demonstrate the ability to deliver a sufficient dose of leuprolide via the oral route of administration to approximate the dose required to also suppress children with CPP into the pre-pubertal range.

### Comparison of Pharmacokinetic Parameters of Oral Leuprolide to Approved Injectable Products

for the Treatment of CPP in Children Presented in Table 3 are relevant PK data from:
1) Fensolvi (leuprolide 45 mg SC 6-month depot), approved for CPP in May 2020,
2) Lupron (1 mg subcutaneous), originally approved for CPP in 1993, and
3) oral leuprolide data from the LOPDT-PH1-02 study of Example 1.

TABLE 3

| Product | Parameter | Value | Reference |
|---|---|---|---|
| Fensolvi 45 mg | Cmax 4 hours | 212.3 ng/mL | Product label and |
| SC 6-month depot | Cplateau 4-48 weeks | 0.37 ng/mL (range | Summary Basis of |
| | | 0.18-0.63 ng/mL) | Approval |
| | AUC 0-6 hours | 37.71 ng × h/mL | |
| | AUC 0-369 days | 113.3 ng × h/mL | |
| Lupron 1 mg SC | Cmax | 32-35 ng/mL | Product label and |
| daily injection | AUC | 118.6 ng × h/mL | Summary Basis of |
| | | | Approval |
| Oral leuprolide | Cmax 0-24 hours | 93.4, 114.8, 230.0, 81.1, | Clinical trial |
| (Treatments A-E) | | 60.7 ng/mL (A-E) | LOPDT-PH1-02 |
| PO daily oral | AUC 0-24 hours | 255.5, 371.6, 534.7, | |
| | | 224.5, 185.6 ng × h/mL | |
| | | (A-E) | |

The PK parameters at Table 3 show that the oral leuprolide dose-regimens tested in adults exceed the corresponding values reported previously for both Fensolvi 6-month depot and Lupron daily SC injection, both of which were shown to be efficacious and were approved for treatment of Central Precocious Puberty in children. It is expected that the pharmacokinetics of oral leuprolide in children will be similar to that in adults. Thus, the leuprolide oral dosage forms and methods of administration described herein are an effective treatment for CPP in children.

### Example 3: Systemic Delivery of Leuprolide Via Oral Administration

The presently disclosed embodiments may include a technology platform employing enteric coating and permeation enhancement to enable systemic delivery of peptides via the oral route. To determine whether the oral formulation described herein enables systemic delivery of therapeutic doses of the GnRH agonist leuprolide via oral route, a randomized, four-period, four-treatment, crossover pharmacokinetic and food effect study was conducted in 14 healthy female volunteers, which compared PK profiles following single administrations of 1 and 4 mg of leuprolide—the latter under both fasting and fed conditions—to that of a single subcutaneous injection of 1 mg of leuprolide acetate.

Study objectives were to assess the relative bioavailability, dose proportionality, safety and tolerability of orally-administered leuprolide, as well as the effect of food on drug absorption. Frequent blood sampling for 24 hours followed each dose, and each treatment was separated by 7 days. Of the 14 subjects enrolled and dosed, 12 completed the study. Mean Tmax occurred at 2.24±0.38, 2.77±0.46, and 3.49±0.23 hrs, for 1 mg po, 4 mg po (fasting), and 1 mg sc leuprolide, respectively. Mean Cmax was 1.9±1.3, 10.2±9.6, and 59.4±9.1 ng/mL, for the aforementioned treatments. Mean AUC0-24h was 3.5±2.1, 19.3±16.8, and 163.0±18.8 ng*h/mL, respectively. Relative to 1 mg se leuprolide, oral doses of 1 mg and 4 mg (fasted) achieved 2.2±1.3% and 3.0±2.5% bioavailability. While differences in the dose-normalized parameters were not statistically significant, formal criterion of dose-proportionality (i.e. 90% CI within 80-125%) was not met. ‘Fasting’ conditions included an overnight (10-hr) fast prior to dosing, and no food consumption for 4 hours following dosing. The ‘fed’ condition involved consumption of a high-fat, high-calorie meal 30 minutes prior to drug administration. Significant negative impact of food on both rapidity and extent of drug absorption was observed in nearly all subjects, with measurable leuprolide levels sufficiently sparse as to prevent robust PK assessments in this group. The subject incidence of treatment-emergent adverse events (TEAEs) was comparable across all four treatment groups, including sc leuprolide, and was not dose-dependent. The most commonly observed TEAEs across all treatment groups combined were nausea (43%), headache (43%), and increased libido (21%). All other TEAEs were reported by only one or two subjects. The study drugs were well tolerated, with most AEs mild in severity, none severe or serious, and 30 of 53 (57%) deemed unrelated to study drug. No notable changes were observed in laboratory values or vital signs.

This study thus confirmed the successful oral delivery of leuprolide, a 1209-Dalton nonapeptide, via the oral route as an alternative to parenteral administration of this widely-used therapeutic.

### Example 4: Effective Suppression of Gonadotropins and Gonadal Steroids Following Oral Administration of Leuprolide

A platform designed to enable systemic delivery of peptides via the oral-GI route was employed to deliver low doses of leuprolide—4 mg qd (Treatment A, n=9), 4 mg bid (Treatment B, n=9), and 10 mg bid (Treatment D, n=12)—daily for 28 days to regularly-menstruating, healthy female volunteers. Pharmacodynamic (PD) effects on LH, FSH, estradiol, and progesterone, along with leuprolide drug levels, were assessed weekly and compared to those following a single intramuscular injection of leuprolide acetate 3.75 mg (Treatment C, n=5) in a parallel-group design. The primary PD endpoint was the subject incidence of E2 suppression to ≤40 pg/mL on Treatment Day 29; additional endpoints—E2 suppression rates on Treatment Days 8, 15 and 22, incidence of ‘ovulation’ defined as progesterone level ≥3 ng/mL, and suppression of LH and FSH levels—were assessed on Treatment Days 8, 15, 22, and 29. Safety and tolerability of the test drugs was also assessed. Dose-dependent suppression of E2 was seen, with 66.7%, 87.5%, 100% and 100% of subjects in Treatments A, B, C and D, respectively, achieving E2≤40 pg/mL. Median E2 levels on days 15, 22, and 29 were <40 pg/mL after 4 mg bid po, and <20 pg/mL after both 10 mg bid po and the 3.75 mg injection. ‘Ovulation rates’ on days 22 and 29 were 0% after both 10 mg bid po and 3.75 mg im. Because of the initial burst following injection of the monthly depot, leuprolide Cmax was greater in Treatment C (17.5±2.7 ng/mL) on Dosing Day 1 than for oral leuprolide (3.7±3.2 ng/mL for 4 mg qd, 4.1±5.1 ng/mL for 4 mg bid), though by Day 29, Css in Treatment C (1.7±1.2 ng/mL) was lower than that day's Cmax for oral leuprolide (2.7±3.6 ng/mL for 4 mg qd, 4.8±6.2 ng/mL for 4 mg bid). Cmax and AUC0-24h were dose-dependent but not strictly dose-proportional. Leuprolide “trough” levels prior to oral dosing were frequently below LLQ (25 pg/mL) in subjects treated with 4 mg qd; this occurred in only 6% of samples on 10 mg bid, and 0% on 4 mg bid, thus confirming reliability of oral delivery of leuprolide. There were no Serious or severe Adverse Events, nor changes in laboratory values of concern. One subject discontinued the study due to a transient episode of nausea, vomiting, and headache during her initial dose of 4 mg po. A total of 80 AEs were treatment-emergent, generally mild in intensity, and spread relatively evenly across all 4 treatment groups.

This study confirms effective suppression of gonadal steroids via oral administration of leuprolide, comparable to that of injectable leuprolide, and some marketed doses of GnRH antagonists (historical data).

1. A method of treating a pediatric subject with central precocious puberty (CPP), the method comprising:
administering orally to the pediatric subject having CPP a total daily dose of from about 20 mg to about 60 mg leuprolide or a pharmaceutically acceptable salt thereof in a solid dosage form, wherein the pediatric subject has a body weight of less than 25 kg.

2. A method of treating a pediatric subject with central precocious puberty (CPP), the method comprising:
administering orally to the pediatric subject having CPP a total daily dose of from about 20 mg to about 40 mg leuprolide or a pharmaceutically acceptable salt thereof in a solid dosage form, wherein the pediatric subject has a body weight of less than 25 kg.

3. A method of treating a pediatric subject with central precocious puberty (CPP), the method comprising:
administering orally to the pediatric subject having CPP a total daily dose of from about 40 mg to about 60 mg leuprolide or a pharmaceutically acceptable salt thereof in a solid dosage form, wherein the pediatric subject has a body weight of less than 25 kg.

4. A method of treating a pediatric subject with central precocious puberty (CPP), the method comprising:
administering orally to the pediatric subject having CPP a total daily dose of from about 40 mg to about 80 mg leuprolide or a pharmaceutically acceptable salt thereof in a solid dosage form, wherein the pediatric subject has a body weight ranging from 25 kg and 37.5 kg.

5. A method of treating a pediatric subject with central precocious puberty (CPP), the method comprising:
administering orally to the pediatric subject having CPP a total daily dose of from about 40 mg to about 60 mg leuprolide or a pharmaceutically acceptable salt thereof in a solid dosage form, wherein the pediatric subject has a body weight ranging from 25 kg and 37.5 kg.

6. A method of treating a pediatric subject with central precocious puberty (CPP), the method comprising:
administering orally to the pediatric subject having CPP a total daily dose of from about 60 mg to about 80 mg leuprolide or a pharmaceutically acceptable salt thereof in a solid dosage form, wherein the pediatric subject has a body weight ranging from 25 kg and 37.5 kg.

7. A method of treating a pediatric subject with central precocious puberty (CPP), the method comprising:
administering orally to the pediatric subject having CPP a total daily dose from about 60 mg to about 120 mg leuprolide or a pharmaceutically acceptable salt thereof in a solid dosage form, wherein the pediatric subject has a body weight of greater than 37.5 kg.

8. A method of treating a pediatric subject with central precocious puberty (CPP), the method comprising:
administering orally to the pediatric subject having CPP a total daily dose from about 60 mg to about 90 mg leuprolide or a pharmaceutically acceptable salt thereof in a solid dosage form, wherein the pediatric subject has a body weight of greater than 37.5 kg.

9. A method of treating a pediatric subject with central precocious puberty (CPP), the method comprising:
administering orally to the pediatric subject having CPP a total daily dose from about 90 mg to about 120 mg leuprolide or a pharmaceutically acceptable salt thereof in a solid dosage form, wherein the pediatric subject has a body weight of greater than 37.5 kg.

10. The method of any one of claims 1 to 9, wherein the total daily dose is administered once daily.

11. The method of claim 10, wherein the total daily dose is administered in the morning before consumption of food.

12. The method of any one of claims 1 to 9, wherein half of the total daily dose is administered twice daily.

13. The method claim 12, wherein a first half and a second half of the daily dose are administered at about 10 hours to about 14 hours interval.

14. The method of claim 12, wherein the first half of the daily dose is administered in the morning before consumption of food.

15. The method claim 12, wherein the second half of the daily dose is administered up to about 6 hours after consumption of food.

16. The method of any one of claims 1 to 9, wherein the administration reduces mean ratio of bone age to chronological age of the subject at the time of measurement to about 1.4 or lower over twelve month treatment period.

17. The method of any one of claims 1 to 9, wherein the administration reduces mean ratio of bone age to chronological age of the subject at the time of measurement to about 1 over about a sixty month treatment period.

18. A method of treating a pediatric subject with central precocious puberty (CPP), the method comprising:
administering orally to the pediatric subject having CPP a solid dosage form comprising a therapeutically effective amount of leuprolide or a pharmaceutically acceptable salt thereof,
wherein the therapeutically effective amount of the leuprolide or pharmaceutically acceptable salt thereof is from about 20 mg to about 120 mg, and
wherein administration reduces mean ratio of bone age to chronological age of the pediatric subject at a time of measurement to about 1.4 or lower over twelve month treatment period.

19. A method of treating a pediatric subject with central precocious puberty (CPP), the method comprising:
administering orally to the pediatric subject having CPP a solid dosage form comprising between about 10 mg to about 60 mg of leuprolide or a pharmaceutically acceptable salt thereof,
wherein administration of the solid dosage form for a suitable period of time reduces mean bone age to chronological age of the pediatric subject at a time of measurement to about 1.4 or lower over about a twelve-month treatment period.

20. The method of claim 18, wherein the solid dosage form comprises from about 20 mg to about 60 mg leuprolide or a pharmaceutically acceptable salt thereof.

21. The method of claim 18, wherein the solid dosage form comprises from about 40 mg to about 60 mg leuprolide or a pharmaceutically acceptable salt thereof.

22. The method of claim 18, wherein the solid dosage form comprises from about 50 mg to about 60 mg leuprolide or a pharmaceutically acceptable salt thereof.

23. The method of claim 18, wherein the solid dosage form comprises about 60 mg of leuprolide or a pharmaceutically acceptable salt thereof.

24. The method of claim 18, wherein the solid dosage form comprises about 50 mg of leuprolide or a pharmaceutically acceptable salt thereof.

25. The method of claim 18, wherein the solid dosage form comprises about 45 mg of leuprolide or a pharmaceutically acceptable salt thereof.

26. The method of claim 18, wherein the solid dosage form comprises more than about 40 mg and less than about 60 mg of leuprolide acetate.

27. The method of any one of claims 18-19, wherein the solid dosage form is administered once daily.

28. The method of any one of claims 18-19, wherein the solid dosage form is administered twice daily.

29. The method of any one of claims 1-9 or 18-19, comprising administering the solid dosage form for a time period of about 6 months, of about 12 months, of about 18 months, of about 24 months, or longer.

30. The method of any one of claims 1-9 or 18-19, wherein the solid dosage form is a tablet or a capsule.

31. The method of claim 30, wherein the solid dosage form is a tablet comprising (a) leuprolide or a pharmaceutically acceptable salt thereof, (b) an absorption enhancer; (c) coated acid particles intermixed with the leuprolide or pharmaceutically acceptable salt thereof, wherein the coating separates the acid from the leuprolide or pharmaceutically acceptable salt thereof in the tablet; (d) an outer layer of an acid-resistant enteric coating effective to transport the tablet through the stomach of the pediatric subject while preventing contact between the leuprolide or pharmaceutically acceptable salt thereof and stomach proteases; and (e) a water soluble barrier layer beneath the outer layer of enteric coating that separates the enteric coating from the coated acid particles.

32. The method of claim 31, wherein the coated acid particles comprise coated citric acid particles.

33. The method of claim 31, wherein the pharmaceutically acceptable salt of leuprolide is leuprolide acetate.

34. The method of claim 31, wherein the absorption enhancer comprises an acyl carnitine.

35. The method of claim 31, wherein the absorption enhancer comprises lauroyl carnitine.

36. The method of any one of claims 1-9 or 18-19, wherein the administration reduces mean ratio of bone age to chronological age of the pediatric subject at a time of measurement to about 1 over about a sixty month treatment period.

37. Leuprolide or a pharmaceutically acceptable salt thereof for use in a method of treating a pediatric subject with central precocious puberty (CPP), the method comprising:
administering orally to the pediatric subject having CPP a total daily dose of from about 20 mg to about 60 mg of the leuprolide or pharmaceutically acceptable salt thereof in a solid dosage form, wherein the pediatric subject has a body weight of less than 25 kg.

38. Leuprolide or a pharmaceutically acceptable salt thereof for use in a method of treating a pediatric subject with central precocious puberty (CPP), the method comprising:
administering orally to the pediatric subject having CPP a total daily dose of from about 20 mg to about 40 mg of the leuprolide or pharmaceutically acceptable salt thereof in a solid dosage form, wherein the pediatric subject has a body weight of less than 25 kg.

39. Leuprolide or a pharmaceutically acceptable salt thereof for use in a method of treating a pediatric subject with central precocious puberty (CPP), the method comprising:
administering orally to a pediatric subject having CPP a total daily dose of from about 40 mg to about 60 mg of the leuprolide or pharmaceutically acceptable salt thereof in a solid dosage form, wherein the pediatric subject has a body weight of less than 25 kg.

40. Leuprolide or a pharmaceutically acceptable salt thereof for use in a method of treating a pediatric subject with central precocious puberty (CPP), the method comprising:
administering orally to the pediatric subject having CPP a total daily dose of from about 40 mg to about 80 mg the leuprolide or pharmaceutically acceptable salt thereof in a solid dosage form, wherein the pediatric subject has a body weight ranging from 25 kg and 37.5 kg.

41. Leuprolide or a pharmaceutically acceptable salt thereof for use in a method of treating a pediatric subject with central precocious puberty (CPP), the method comprising:
administering orally to the pediatric subject having CPP a total daily dose of from about 40 mg to about 60 mg of the leuprolide or pharmaceutically acceptable salt thereof in a solid dosage form, wherein the pediatric subject has a body weight ranging from 25 kg and 37.5 kg.

42. Leuprolide or a pharmaceutically acceptable salt thereof for use in a method of treating a pediatric subject with central precocious puberty (CPP), the method comprising:
administering orally to the pediatric subject having CPP a total daily dose of from about 60 mg to about 80 mg of the leuprolide or pharmaceutically acceptable salt thereof in a solid dosage form, wherein the pediatric subject has a body weight ranging from 25 kg and 37.5 kg.

43. Leuprolide or a pharmaceutically acceptable salt thereof for use in a method of treating a pediatric subject with central precocious puberty (CPP), the method comprising:
administering orally to the pediatric subject having CPP a total daily dose from about 60 mg to about 120 mg of the leuprolide or pharmaceutically acceptable salt thereof in a solid dosage form, wherein the pediatric subject has a body weight of greater than 37.5 kg.

44. Leuprolide or a pharmaceutically acceptable salt thereof for use in a method of treating a pediatric subject with central precocious puberty (CPP), the method comprising:
administering orally to the pediatric subject having CPP a total daily dose from about 60 mg to about 90 mg of the leuprolide or a pharmaceutically acceptable salt thereof in a solid dosage form, wherein the pediatric subject has a body weight of greater than 37.5 kg.

45. Leuprolide or a pharmaceutically acceptable salt thereof for use in a method of treating a pediatric subject with central precocious puberty (CPP), the method comprising:
administering orally to the pediatric subject having CPP a total daily dose from about 90 mg to about 120 mg of the leuprolide or pharmaceutically acceptable salt thereof in a solid dosage form, wherein the pediatric subject has a body weight of greater than 37.5 kg.

46. Leuprolide or a pharmaceutically acceptable salt thereof for use in a method of treating a pediatric subject with central precocious puberty (CPP), the method comprising:
administering orally to the pediatric subject having CPP a solid dosage from comprising a therapeutically effective amount of leuprolide or a pharmaceutically acceptable salt thereof,
wherein the therapeutically effective amount of the leuprolide or pharmaceutically acceptable salt thereof is from about 20 mg to about 120 mg, and
wherein administration reduces mean ratio of bone age to chronological age of the pediatric subject at a time of measurement to about 1.4 or lower over twelve month treatment period.

47. Leuprolide or a pharmaceutically acceptable salt thereof for use in a method of treating a pediatric subject with central precocious puberty (CPP), the method comprising:
administering orally to the pediatric subject having CPP a solid dosage form comprising between about 10 mg to about 60 mg of the leuprolide or pharmaceutically acceptable salt thereof,
wherein administration of the solid dosage form for a suitable period of time reduces mean bone age to chronological age of the pediatric subject at a time of measurement to about 1.4 or lower over about a twelve-month treatment period.
